(19) United States
(12) Patent Application Publication
(10) Pub. No.: US 2016/0207988 A1
(43) Pub. Date: Jul. 21, 2016

(54) A NOVEL HORMONE THAT PROMOTES BONE RESORPTION AND USES THEREOF

(71) Applicant: THE BOARD OF REGENTS OF THE UNIVERSITY OF TEXAS SYSTEM, Austin, TX (US)

(72) Inventor: Yihong WAN, Coppell, TX (US)

(73) Assignee: The Board of Regents of the University of Texas System, Austin, TX (US)

(21) Appl. No.: 15/024,357

(22) PCT Filed: Sep. 23, 2014
(86) PCT No.: PCT/US14/56934

§ 371 Date: Mar. 23, 2016

Related U.S. Application Data
(60) Provisional application No. 61/881,672, filed on Sep. 24, 2013.

(51) Int. Cl. C07K 16/18; A61K 45/06; A61K 38/18

(52) U.S. Cl. CPC C07K 16/18; A61K 38/1825; A61K 45/06; A61K 2039/505

(57) ABSTRACT

The disclosure describes the use of anti-IGFBP1 antibodies to inhibit the bone loss-inducing properties of FGF21. This can be applied in the context of FGF21 treatment for obesity and/or diabetes, or in menopause-induced bone loss, such as osteoporosis.

This application claims benefit of priority to U.S. Provisional Application Ser. No. 61/881,672, filed Sep. 24, 2013, the entire contents of which are hereby incorporated by reference.

### FEDERAL GOVERNMENT SUPPORT CLAUSE

This invention was made with government support under grant no. RO1DK089113 awarded by the National Institutes of Health. The Government has certain rights in the invention.

### BACKGROUND

Osteoclasts, the professional bone resorbing cells, are essential for bone turnover and skeletal regeneration. However, excessive osteoclast activity can lead to diseases such as osteoporosis and cancer bone metastasis. Thus, the maintenance of bone mass and bone quality relies on the balanced acts between osteoclast-mediated bone resorption and osteoblast-mediated bone formation. Osteoclastogenesis, the differentiation of osteoclasts from hematopoietic progenitors in response to receptor activator of nuclear factor kappa-B ligand (RANKL), is regulated by not only local factors but also systemic elements (Novak and Teitelbaum, 2008). For examples, endocrine hormones such as parathyroid hormone (PTH) from the parathyroid gland and calcitonin from the thyroid gland promote or suppress bone resorption, respectively. Moreover, osteoclastogenesis can also be stimulated by pharmacological agents such as rosiglitazone, a widely used drug for diabetes and insulin resistance (Wan et al., 2007).

Fibroblast growth factor 21 (FGF21) is a powerful regulator of glucose and lipid metabolism (Potthoff et al., 2012). FGF21 administration to diabetic mice and rhesus monkeys strongly enhances insulin sensitivity, decreases plasma glucose and triglyceride, and reduces body weight (Berglund et al., 2009, Coskun et al., 2008, Kharitonenkov et al., 2005 and Xu et al., 2009). Thus, FGF21 is a potential new drug for the treatment of obesity and diabetes that is currently in clinical trials. The inventor has recently identified FGF21 as a potent negative regulator of skeletal mass and a key integrator of bone and energy metabolism (Wei et al., 2012). Both genetic and pharmacologic FGF21 gain-of-function leads to a decrease in bone mass. In contrast, FGF21 loss-of-function leads to a reciprocal high-bone-mass. This finding suggests that skeletal fragility may be an undesirable consequence of chronic FGF21 administration. Therefore, the identification of the cellular and molecular mechanisms for how FGF21 controls bone homeostasis may illuminate new strategies to addresses its detrimental bone loss side effects.

### SUMMARY

Thus, in accordance with the present disclosure, there is provided a method of treating menopause-induced bone loss in a subject comprising administering to a subject in need thereof an anti-IGFBP1 antibody. The antibody may be part of a polyclonal antibody preparation, or may be a monoclonal antibody. The antibody may be a humanized antibody, a chimeric antibody, an antibody fragment, a bispecific antibody or a single chain antibody. The antibody may be administered systemically, or administered local or regional to a site of bone loss or potential bone loss. The subject may be a human subject or a non-human animal subject. The menopause may be age-related or surgery-related.

There also is provided a method of treating a subject for bone resorption induced by FGF21 therapy comprising administering to a subject in need thereof an anti-IGFBP1 antibody. The antibody may be part of a polyclonal antibody preparation, or may be a monoclonal antibody. The antibody may be a humanized antibody, a chimeric antibody, an antibody fragment, a bispecific antibody or a single chain antibody. The antibody may be administered systemically, or administered local or regional to a site of bone resorption or potential bone resorption. The subject may be a human subject or a non-human animal subject. The antibody may be co-administered with FGF21 or administered before or after FGF21.

In another embodiment, there is provided a method of treating FGF21-induced bone loss/resorption comprising administering to a subject in need thereof an integrin β-1 inhibitor. The FGF21-induced bone loss/resorption may be menopause-related. The menopause may be age-related or surgery-related. The FGF21-induced bone loss/resorption may be FGF21 therapy-related. The inhibitor may be co-administered with FGF21 or administered before or after FGF21. The inhibitor may be an anti-integrin β-1 antibody, an integrin β-1 siRNA, an integrin β-1 fragment or sharpin. The inhibitor may be administered systemically, or administered local or regional to a site of bone loss/resorption or potential bone loss/resorption. The subject may be a human subject or a non-human animal subject. Embodiments discussed in the context of methods and/or compositions of the disclosure may be employed with respect to any other method or composition described herein. Thus, an embodiment pertaining to one method or composition may be applied to other methods and compositions of the disclosure as well.

As used herein the specification, “a” or “an” may mean one or more. As used herein in the claim(s), when used in conjunction with the word “comprising”, the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

Throughout this application, the term “about” is used to indicate that a value includes the inherent variation of error for the device, the method being employed to determine the value, or the variation that exists among the study subjects.

Other objects, features and advantages of the present disclosure will become apparent from the following detailed description. It should be understood, however, that the detailed description and the specific examples, while indicating preferred embodiments of the disclosure, are given by way of illustration only, since various changes and modifications within the spirit and scope of the disclosure will become apparent to those skilled in the art from this detailed description.

### BRIEF DESCRIPTION OF THE DRAWINGS

The following drawings form part of the present specification and are included to further demonstrate certain aspects of the present disclosure. The disclosure may be better understood by reference to one or more of these drawings in combination with the detailed description of specific embodiments presented herein.

FIGS. 1A-H. Liver β-Klotho Deletion Decreases Bone Resorption. (FIGS. 1A-D) Serum CTX-1 bone resorption marker in liver β-Klotho KO (L-Klb-KO) (FIG. 1A), fat β-Klotho KO (F-Klb-KO) (FIG. 1B), CamK-Cre-mediated brain β-Klotho KO (Cam-Klb-KO) (FIG. 1C), Nestin-Cre-mediated brain β-Klotho KO (Nes-Klb-KO) (FIG. 1D) or corresponding littermate control mice. All mice were 3-4 month old males (n=4). (FIGS. 1E-G) L-Klb-KO mice displayed a high-bone-mass phenotype. Tibiae from L-Klb-KO or WT littermate controls (4 month old, male, n=4) were analyzed by μCT. (FIG. 1E) Representative images of the trabecular bone of the tibial metaphysis (top) (scale bar, 10 μm) and the entire proximal tibia (bottom) (scale bar, 1 mm) (FIG. 1F) Quantification of trabecular bone volume and architecture. BV/TV, bone volume/tissue volume ratio; BS, bone surface; BS/BV, bone surface/bone volume ratio; Tb.N, trabecular number; Tb.Th, trabecular thickness; Tb.Sp, trabecular separation. (FIG. 1G) Cortical bone BV/TV. (FIG. 1H) Serum P1NP bone formation marker in L-Klb-KO mice or WT control mice (4 month old, male, n=4). Statistical analyses were performed with Student's t-Test and are shown as mean±standard deviation; *, p<0.05; ***, p<0.005; n.s. non-significant (p>0.05).

FIGS. 2A-G. Liver β-Klotho Deletion Abolishes FGF21-Induced Bone Loss. (FIGS. 2A-F) WT or L-Klb-KO mice (6-month-old at end point, male, n=4) were fed a high-fat-diet for 4.5 months, and then treated with FGF21 (1 mg/kg/day) or vehicle control daily by intraperitoneal (IP) injection for the last 14 days. (FIGS. 2A-B) μCT of the tibiae showed that FGF21-induced bone loss was abolished in the L-Klb-KO mice. (FIG. 2A) Representative images of the trabecular bone of the tibial metaphysis (top) (scale bar, 10 μm) and the entire proximal tibia (bottom) (scale bar, 1 mm) (FIG. 2B) Quantification of trabecular bone volume and architecture. BV/TV, bone volume/tissue volume ratio; BS, bone surface; Tb.N, trabecular number; Tb.Sp, trabecular separation. (FIG. 2C) Serum CTX-1 bone resorption marker. (FIG. 2D) Serum P1NP bone formation marker. (FIG. 2E) Plasma insulin. (FIG. 2F) Plasma glucose. Statistical analyses were performed with Student's t-Test and are shown as mean±standard deviation; black * compares FGF21 treatment with vehicle control in the same genotype; blue ⁺ compares L-Klb-KO with WT control in the same treatment; *, p<0.05; **, p<0.01; ***, p<0.005; ****, p<0.001; n.s. non-significant (p>0.05). (FIG. 2G) Insulin tolerance test. WT or L-Klb-KO mice (5-month-old, male, n=6) were fasted for 4 hrs, and then received a single IP injection of FGF21 (1 mg/kg) or vehicle control, co-injected with insulin (0.75 U/kg). Statistical analyses were performed with Student's t-Test and are shown as mean±standard error; * compares FGF21 treatment with vehicle control in the same genotype; *, p<0.05; ***, p<0.005; ****, p<0.001.

FIGS. 3A-N. IGFBP1 is an FGF21-Induced Pro-Osteoclastogenic Hepatokine. (FIGS. A-C) Effects of liver conditioned media (LCM) on osteoclast differentiation from WT bone marrow cells, quantified by the mRNA expression of the osteoclast marker TRAP (n=4); * compares LCM treatment with mock controls. (FIG. 3A) LCM from WT or FGF21-Tg mice (2-month-old, male, n=4) compared to mock control. R, RANKL; Rosi, rosiglitazone. (FIG. 3B) LCM from WT or L-Klb-KO mice (4-month-old, male, n=4) compared to mock control. (FIG. 3C) LCM from WT mice (3-month-old, male, n=3) treated with FGF21 (1 mg/kg) or PBS vehicle control for 1 hr. (FIG. 3D) IGFBP1 mRNA expression in the liver and tibia (bone+marrow) from WT and FGF21-Tg mice (n=3); n.d.=no detection. (FIG. 3E) Liver IGFBP1 mRNA expression in total β-Klotho KO mice (T-Klb-KO) or total β-Klotho heterozygous controls (T-Klb-Het) with or without FGF21-Tg allele (6-month-old, male, n=6); * compares FGF21-Tg with WT; ⁺ compares T-Klb-KO with T-Klb-Het. (FIG. 3F) Liver IGFBP1 mRNA expression in L-Klb-KO mice or WT controls treated with FGF21 (1 mg/kg) or PBS vehicle control for 1 hr (3-month-old, male, n=3); * compares FGF21 with vehicle; ⁺ compares L-Klb-KO with WT control. (FIG. 3G) IGFBP1 protein levels in the serum (top) and liver (bottom) of WT and FGF21-Tg mice (2-month-old, male, n=4). (FIG. 3H) IGFBP1 protein levels in the serum (top) and liver (bottom) of WT and L-Klb-KO mice that were fasted for 24 hr (10-month-old, male, n=3). (FIG. 3I) The pro-osteoclastogenic activity of WT LCM was abolished by an IGFBP1-blocking antibody (anti-IGFBP1, 100 ng/ml), quantified by TRAP mRNA expression in WT bone marrow osteoclast differentiation cultures treated with vehicle control (V), RANKL (R), or RANKL+rosiglitazone (R+Rosi) (n=3). IgG served as a negative control. (FIGS. 3J-K) Recombinant mouse IGFBP1 enhanced the RANKL-mediated and rosiglitazone-stimulated osteoclast differentiation from WT bone marrow cells in a dose-dependent manner. (FIG. 3J) Quantification of TRAP mRNA expression (n=3); ⁺ compares IGFBP1 treatment with no IGFBP1 controls. (FIG. 3K) Representative images of TRAP-stained differentiation cultures showing that IGFBP1 increased the number and size of mature osteoclasts. Mature osteoclasts were identified as multinucleated (>3 nuclei) TRAP⁺ (purple) cells. Scale bar, 25 μm. (FIG. 3L) Osteoclast differentiation from RAW264.7 mouse macrophage cell line was induced by RANKL and further enhanced by mouse and human IGFBP1, quantified by TRAP mRNA (n=3). (FIGS. 3M-N) Osteoclast differentiation from human peripheral blood mononuclear (hPBMN) cells was induced by human RANKL and further enhanced by human IGFBP1. (FIG. 3M) Quantification of TRAP mRNA expression on day 9 (n=3). (FIG. 3N) Representative images of TRAP-stained differentiation cultures on day 14. Mature osteoclasts were identified as multinucleated (>3 nuclei) TRAP⁺ (purple) cells. Scale bar, 25 μm. Statistical analyses were performed with Student's t-Test and are shown as mean±standard error; *, p<0.05; **, p<0.01; ***, p<0.005; ****, p<0.001.

FIGS. 4A-O. Pharmacological IGFBP1 Manipulation Alters Bone Resorption In Vivo. (FIGS. 4A-E) Effects of IGFBP1 and anti-IGFBP1 treatment on bone. WT C57B6 mice (10-week-old, male, n=4) were treated with vehicle control (Veh), recombinant mouse IGFBP1 (0.015 mg/kg/day) or anti-IGFBP1 (0.03 mg/kg/day) by daily IP injection for 14 days. (FIG. 4A) Serum CTX-1 bone resorption marker. (FIG. 4B) Serum P1NP bone formation marker. (FIG. 4C) Representative μCT images of the entire proximal tibia (scale bar, 1 mm) (FIG. 4D) Quantification of trabecular bone volume and architecture. BV/TV, bone volume/tissue volume ratio; BS, bone surface; Tb.N, trabecular number; Tb.Sp, trabecular separation. (FIGS. 4F-I) Anti-IGFBP1 treatment attenuates ovariectomy (OVX)-induced bone resorption and bone loss. WT C57B6 mice (18-week-old, female) were ovariectomized or sham-operated. Three days after surgery, they were treated with anti-IGFBP1 (anti-BP1) or IgG control at 0.03 mg/kg/day and 3 times/week for 5 weeks (n=5). (FIG. 4F) Uterine weight. (FIG. 4G) Serum CTX-1 bone resorption marker. (FIG. 4H) Serum P1NP bone formation marker. (FIG. 4I) BV/TV by μCT. (FIGS. 4J-N) Anti-IGFBP1 treatment abolishes FGF21-induced bone resorption and bone loss. (FIGS. 4J-L) All mice were 7-8 month old females on chow diet (n=4). FGF21-Tg mice were treated with anti-IGFBP1 (anti-BP1) or IgG control at 0.03 mg/kg/day daily for 14 days. Serum CTX-1 (FIG. 4J), serum P1NP (FIG. 4K), trabecular BV/TV and BS in proximal tibiae (I) were compared with WT mice or untreated FGF21-Tg mice. (FIGS. 4M-O) All mice were 9-10 month old females that were on high-fat-diet for 5 weeks (n=5). FGF21-Tg mice were treated with anti-IGFBP1 at 0.03 mg/kg/day daily for the last 14 days. Serum CTX-1 (FIG. 4M) and serum P1NP (FIG. 4N) were compared with untreated FGF21-Tg mice or WT mice. (FIG. 4O) Anti-IGFBP1 treatment did not affect the insulin-sensitizing effect of FGF21. FGF21-Tg mice or WT controls were treated as in (FIGS. 4M-N) and subjected to insulin tolerance test (n=5). Statistical analyses were performed with Student's t-Test and are shown as mean±standard error; *, p<0.05; **, p<0.01; ***, p<0.005; ****, p<0.001.

FIGS. 5A-F. IGFBP1 Potentiates RANKL Signaling by Enhancing Erk-phosphorylation and NFATc1 Activation. (FIG. 5A) A schematic diagram of the time course of the bone marrow osteoclast differentiation assay. d, day. (FIG. 5B) IGFBP1 functions at the RANKL-induced differentiation stage. IGFBP1 were added to WT osteoclast differentiation cultures on day 1-3 only, day 4-6 only, or the entire day 1-6; osteoclast differentiation was quantified by the mRNA expression of TRAP (n=3). (FIG. 5C) IGFBP1 does not affect cell proliferation in the osteoclast differentiation culture, measured by MTT assays. (FIG. 5D) IGFBP1 induces ERK phosphorylation in synergy with RANKL. WT bone marrow cells were cultured with MCSF for 5 days, in the absence or presence of RANKL during the last 2 days. The cells were treated with IGFBP1 (50 ng/ml) for the indicated amount of time, the levels of p-ERK and total ERK (t-ERK) were analyzed by western blot. The p-ERK/t-ERK ratio was quantified as fold changes compared to lane 1. (FIG. 5E) IGFBP1 does not affect basal or RANKL-induced c-Jun phosphorylation (Ser73) or NFκB degradation. WT bone marrow cells were cultured with MCSF for 3 days, and then treated with IGFBP1 (50 ng/ml) and/or RANKL (100 ng/ml) for the indicated amount of time. (FIG. 5F) IGFBP1 specifically enhances RANKL-induced NFATc1 activation. RAW264.7 cells were transfected with NFATc1-Luc, AP1-Luc or NFκB-Luc reporter together with a CMV-βgal reporter as internal control for 24 hrs, and then treated with RANKL and/or IGFBP1 for 24 hrs before reporter assays (n=5). Statistical analyses were performed with Student's t-Test and are shown as mean±standard error; *, p<0.05; ****, p<0.001; n.s. non-significant (p>0.05).

FIGS. 6A-M. IGFBP1 Functions via RGD Binding to Integrin β1 Receptor. (FIG. 6A) Amino acid sequence alignment of the C-termini of IGFBP1 from human, pig, mouse and rat. The conserved amino acids are highlighted in red, the RGD motifs are highlighted in yellow. (FIG. 6B) An RGD-containing peptide dose-dependently abolished the pro-osteoclastogenic activity of IGFBP1, quantified by TRAP mRNA expression (n=3). (FIG. 6C) IGFBP1-induced ERK phosphorylation was abolished by RGD-containing peptide or anti-IGFBP1. WT bone marrow cells were cultured with MCSF for 5 days, and then pretreated with RGD-containing peptide (20 μg/ml) or anti-IGFBP1 (0.5 μg/ml) for 15 min before treating with IGFBP1 (50 ng/ml) for 5 min. The p-ERK/t-ERK ratio was quantified as fold changes compared to lane 1. (FIG. 6D) Itgb1 mRNA expression was diminished in osteoclast precursors from the bone marrow of Oc-Itgb1-KO mice compared with littermate WT control mice on day 3 of culture with MCSF (n=3). (FIGS. 6E-F) IGFBP1 enhancement of osteoclast differentiation was completely abolished in the cultures from the bone marrow of Oc-Itgb1-KO mice compared with littermate WT control mice (n=3). (FIG. 6E) Expression of a representative osteoclast marker TRAP. (FIG. 6F) Representative images of TRAP-stained differentiation cultures showing the number and size of mature osteoclasts. Mature osteoclasts were identified as multinucleated (>3 nuclei) TRAP+ (purple) cells. Scale bar, 25 μm. (FIGS. 6G-H) IGFBP1 induction of bone resorption and bone loss was abolished in Oc-Itgb1-KO mice. Oc-Itgb1-KO mice or WT littermate controls (2-month-old males, n=4) were treated with IGFBP1 (0.015 mg/kg/day) or PBS for 14 days. (FIG. 6G) Serum CTX-1. (FIG. 6H) μCT analysis of BV/TV and Tb.N. (FIG. 6I) IGFBP1 induction of Erk phosphorylation was abolished in Oc-Itgb1-KO mice. (FIGS. 6J-L) Osteoclastic Itgb1 deletion in the FGF21-Tg mice attenuates FGF21-induced bone loss while retains FGF21-induce insulin sensitization. FGF21-Tg; Oc-Itgb1-KO compound mutants were compared with littermate WT, FGF21-Tg or Oc-Itgb1-KO mice (3 month old males, n=4) after 4 weeks of high-fat-diet feeding. (FIG. 6J) Serum CTX-1. (FIG. 6K) BV/TV by μCT. (FIG. 6L) ITT assay. (FIG. 6M) A simplified model for how the liver hormone IGFBP1 regulates osteoclast differentiation and mediates FGF21-induced bone resorption. FGF21 activation of its co-receptor β-Klotho induces the expression and secretion of IGFBP1 from the liver. IGFBP1 in turn functions as an endocrine hormone by binding to its receptor integrin α5β1 on the osteoclast precursors, leading to the potentiation of RANKL signaling and accelerated osteoclast differentiation. Consequently, a physiological or pharmacological elevation of FGF21 level results in a higher circulating IGFBP1 level, leading to increased bone resorption and bone loss. Statistical analyses were performed with Student's t-Test and are shown as mean±standard error; *, p<0.05; **, p<0.01; ***, p<0.005; ****, p<0.001.

FIGS. 7A-B. IGFBP1 does not affect osteoblast differentiation or RANKL/OPG expression. (FIGS. 7A-B) Bone marrow osteoblast differentiation cultures were treated with 0 or 10 ng/ml of IGFBP1. (FIG. 7A) Osteoblast differentiation was unaltered, quantified by the mRNA expression of osteoblast markers osteocalcin and Collal (n=3). ObDiff, osteoblast differentiation cocktail. (FIG. 7B) RANKL and OPG expression in osteoblasts were unaltered by IGFBP1 (n=3).

### DETAILED DESCRIPTION

The inventor previously found that FGF21 induces bone loss by simultaneously decreasing bone formation and increasing bone resorption (Wei et al., 2012). However, how FGF21 enhances bone resorption was unknown. Their previous data show that FGF21 does not directly regulate osteoclast differentiation from bone marrow hematopoietic progenitors (Wei et al., 2012). Consistent with this observation, the FGF21 co-receptor β-Klotho is not expressed in the macrophage/osteoclast lineage (Wei et al., 2012 and Ding et al., 2012). These results indicate that FGF21 acts on other cell types and tissues to indirectly promote osteoclastogenesis and bone resorption.

Unexpectedly, here the inventor shows that liver-specific deletion of the FGF21 co-receptor β-Klotho attenuates both physiological and FGF21-stimulated bone resorption. The inventor detected a pro-osteoclastogenic activity in the hepatic secretome that is increased by FGF21 but decreased by β-Klotho deletion, and largely attributed to insulin-like growth factor binding protein 1 (IGFBP1). Recombinant IGFBP1 enhances, whereas an IGFBP1-blocking antibody suppresses, osteoclast differentiation and bone resorption. Moreover, anti-IGFBP1 treatment effectively attenuates ovariectomy-induced bone resorption, indicating that IGFBP1 blockade may be a new therapeutic strategy for postmenopausal osteoporosis. Intriguingly, anti-IGFBP1 treatment also diminishes FGF21-induced bone resorption and bone loss without compromising its insulin-sensitizing effects, indicating that IGFBP1 blockade may be an exciting avenue to ameliorate the skeletal side effects while retaining the metabolic benefits of FGF21. Mechanistically, IGFBP1 functions via its RGD domain to bind to its receptor integrin α5β1 on osteoclast precursors, hence potentiating RANKL-stimulated erk-phosphorylation and NFATc1 activation. Therefore, the hepatokine IGFBP1 is a novel yet critical liver-bone endocrine relay that promotes osteoclastogenesis and bone resorption.

### I. FGF21

Fibroblast growth factor 21 is a protein that in humans is encoded by the FGF21 gene. The protein encoded by this gene is a member of the fibroblast growth factor (FGF) family. FGF family members possess broad mitogenic and cell survival activities and are involved in a variety of biological processes including embryonic development, cell growth, morphogenesis, tissue repair, tumor growth and invasion. FGF21 is specifically induced by HMGCS2 activity. The oxidized form of ketone bodies (acetoacetate) in a cultured medium also induced FGF21, possibly via a SIRT1-dependent mechanism. HMGCS2 activity has also been shown to be increased by deacetylation of lysines 310, 447, and 473 via SIRT3 in the mitochondria.

In mice, brown adipose tissue becomes a source of systemic FGF21 after cold exposure. Norepinephrine, acting via β-adrenergic, cAMP-mediated, mechanisms and subsequent activation of protein kinase A and p38 MAPK, induces FGF21 gene transcription and also FGF21 release in brown adipocytes. ATF2 binding to the FGF21 gene promoter mediates cAMP-dependent induction of FGF21 gene transcription. Release of FGF21 by brown fat in vivo was accompanied by a reduction in systemic FGF21 half-life. LXR represses FGF21 in humans via an LXR response element located from −37 to −22 by on the human FGF21 promoter.

FGF21 stimulates glucose uptake in adipocytes but not in other cell types. This effect is additive to the activity of insulin. FGF21 treatment of adipocytes is associated with phosphorylation of FRS2, a protein linking FGF receptors to the Ras/MAP kinase pathway. FGF21 injection in ob/ob mice results in an increase in Glut1 in adipose tissue. FGF21 also protects animals from diet-induced obesity when overexpressed in transgenic mice and lowers blood glucose and triglyceride levels when administered to diabetic rodents. Treatment of animals with FGF21 results in increased energy expenditure, fat utilization and lipid excretion. Beta Klotho (KLB) functions as a cofactor essential for FGF21 activity.

In cows plasma FGF21 was nearly undetectable in late pregnancy, peaked at parturition, and then stabilized at lower, chronically elevated concentrations during early lactation. Plasma FGF21 was similarly increased in the absence of parturition when an energy-deficit state was induced by feed restricting late-lactating dairy cows, implicating energy insufficiency as a cause of chronically elevated FGF21 in EL. The liver was the major source of plasma FGF21 in early lactation with little or no contribution by WAT, skeletal muscle, and mammary gland. Meaningful expression of the FGF21 coreceptor β-Klotho was restricted to liver and WAT in a survey of 15 tissues that included the mammary gland. Expression of β-Klotho and its subset of interacting FGF receptors was modestly affected by the transition from LP to EL in liver but not in WAT.

Serum FGF-21 levels were significantly increased in patients with type 2 diabetes mellitus (T2DM) which may indicate a role in the pathogenesis of T2DM. Elevated levels also correlate with liver fat content in non-alcoholic fatty liver disease and positively correlate with BMI in humans suggesting obesity as a FGF21-resistant state. Mice lacking FGF21 fail to fully induce PGC-1α expression in response to a prolonged fast and have impaired gluconeogenesis and ketogenesis.

FGF21 stimulates phosphorylation of fibroblast growth factor receptor substrate 2 and ERK1/2 in the liver. Acute FGF21 treatment induced hepatic expression of key regulators of gluconeogenesis, lipid metabolism, and ketogenesis including glucose-6-phosphatase, phosphoenol pyruvate carboxykinase, 3-hydroxybutyrate dehydrogenase type 1, and carnitine palmitoyltransferase 1α. In addition, injection of FGF21 was associated with decreased circulating insulin and free fatty acid levels. FGF21 treatment induced mRNA and protein expression of PGC-1α, but in mice PGC-1α expression was not necessary for the effect of FGF21 on glucose metabolism.

In mice FGF21 is strongly induced in liver by prolonged fasting via PPAR-α and in turn induces the transcriptional coactivator PGC-1α and stimulates hepatic gluconeogenesis, fatty acid oxidation, and ketogenesis. FGF21 also blocks somatic growth and sensitizes mice to a hibernation-like state of torpor, playing a key role in eliciting and coordinating the adaptive starvation response. FGF21 expression is also induced in white adipose tissue by PPAR-γ, which may indicate it also regulates metabolism in the fed state.

Activation of AMPK and SIRT1 by FGF21 in adipocytes enhanced mitochondrial oxidative capacity as demonstrated by increases in oxygen consumption, citrate synthase activity, and induction of key metabolic genes. The effects of FGF21 on mitochondrial function require serine/threonine kinase 11 (STK11/LKB1), which activates AMPK Inhibition of AMPK, SIRT1, and PGC-1α activities attenuated the effects of FGF21 on oxygen consumption and gene expression, indicating that FGF21 regulates mitochondrial activity and enhances oxidative capacity through an LKB1-AMPK-SIRT1-PGC-1α-dependent mechanism in adipocytes, resulting in increased phosphorylation of AMPK, increased cellular NAD+ levels and activation of SIRT1 and deacetylation of SIRT1 targets PGC-1α and histone 3.

The accession number for the mRNA sequences of FGF21 is NM_019113, while the protein is NM_061986.

### II. IGFBP1

Insulin-like growth factor-binding protein 1 (IBP-1) also known as placental protein 12 (PP12) is a protein that in humans is encoded by the IGFBP1 gene. This gene is a member of the Insulin-like growth factor-binding protein (IGFBP) family and encodes a protein with an IGFBP domain and a type-I thyroglobulin domain. The protein binds both insulin-like growth factors (IGFs) I and II and circulates in the plasma. Binding of this protein prolongs the half-life of the IGFs and alters their interaction with cell surface receptors. Alternate transcriptional splice variants, encoding different isoforms, have been characterized.

The accession number for the mRNA sequences of IGFBP1 is NM_000596, while the protein is NM_000587.

### III. Integrin β1

Integrins are transmembrane receptors that are the bridges for cell-cell and cell-extracellular matrix (ECM) interactions. When triggered, integrins in turn trigger chemical pathways to the interior (signal transduction), such as the chemical composition and mechanical status of the ECM, which results in a response (activation of transcription) such as regulation of the cell cycle, cell shape, and/or motility; or new receptors being added to the cell membrane. This allows rapid and flexible responses to events at the cell surface, for example to signal platelets to initiate an interaction with coagulation factors. Integrins work alongside other receptors such as cadherins, the immunoglobulin superfamily cell adhesion molecules, selectins and syndecans to mediate cell-cell and cell-matrix interaction. Ligands for integrins include fibronectin, vitronectin, collagen, and laminin.

There are several types of integrins, and a cell may have several types on its surface. Integrins have been found in all animals investigated, from sponges to mammals. Integrins have two different chains, the α (alpha) and β (beta) subunits, and are called obligate heterodimers. In mammals, there are eighteen α and eight β subunits, in Drosophila five α and two β subunits, and in Caenorhabditis nematodes two α subunits and one β subunit. The α and β subunits each penetrate the plasma membrane and possess small cytoplasmic domains.

β-1 integrin associates with a subunits to form receptor complexes that bind a variety of ligands. Integrins α-1/β-1, α-2/β-1, α-10/β-1 and α-11/β-1 are receptors for collagen. Integrins α-2/β-1, α-3/α-1, α-4/α-1, α-5/α-1, α-8/β-1, α-10/β-1, α-11/β-1 and α-V/β-1 are receptors for fibronectin. Integrin alpha-5/beta-1 is a receptor for fibrinogen. Integrin α-1/β-1, α-2/β-1, α-6/β-1 and α-7/β-1 are receptors for lamimin. α-7/β-1 integrin regulates cell adhesion and laminin matrix deposition. Integrin α-4/β-1 is a receptor for VCAM1. Integrin α-9/β-1 is a receptor for VCAM1, cytotactin and osteopontin. Integrin α-3/β-1 is a receptor for epiligrin, thrombospondin and CSPG4 and has a role in endothelial cell motility and angiogenesis.

The accession number for the integrin β-1 protein sequence is CAA68738.1, and the for mRNA is P09055.

### IV. Antibodies and Antibody Production

### A. General Methods

Antibodies to the MUC1-C/ECD may be produced by standard methods as are well known in the art (see, e.g., Antibodies: A Laboratory Manual, Cold Spring Harbor Laboratory, 1988; U.S. Pat. No. 4,196,265). The methods for generating monoclonal antibodies (MAbs) generally begin along the same lines as those for preparing polyclonal antibodies. The first step for both these methods is immunization of an appropriate host or identification of subjects who are immune due to prior natural infection. As is well known in the art, a given composition for immunization may vary in its immunogenicity. It is often necessary therefore to boost the host immune system, as may be achieved by coupling a peptide or polypeptide immunogen to a carrier. Exemplary and preferred carriers are keyhole limpet hemocyanin (KLH) and bovine serum albumin (BSA). Other albumins such as ovalbumin, mouse serum albumin or rabbit serum albumin can also be used as carriers. Means for conjugating a polypeptide to a carrier protein are well known in the art and include glutaraldehyde, m-maleimidobencoyl-N-hydroxysuccinimide ester, carbodiimide and bis-biazotized benzidine. As also is well known in the art, the immunogenicity of a particular immunogen composition can be enhanced by the use of non-specific stimulators of the immune response, known as adjuvants. Exemplary and preferred adjuvants include complete Freund's adjuvant (a non-specific stimulator of the immune response containing killed Mycobacterium tuberculosis), incomplete Freund's adjuvants and aluminum hydroxide adjuvant. The amount of immunogen composition used in the production of polyclonal antibodies varies upon the nature of the immunogen as well as the animal used for immunization. A variety of routes can be used to administer the immunogen (subcutaneous, intramuscular, intradermal, intravenous and intraperitoneal). The production of polyclonal antibodies may be monitored by sampling blood of the immunized animal at various points following immunization. A second, booster injection, also may be given. The process of boosting and titering is repeated until a suitable titer is achieved. When a desired level of immunogenicity is obtained, the immunized animal can be bled and the serum isolated and stored, and/or the animal can be used to generate MAbs.

Following immunization, somatic cells with the potential for producing antibodies, specifically B lymphocytes (B cells), are selected for use in the MAb generating protocol. These cells may be obtained from biopsied spleens or lymph nodes, or from circulating blood. The antibody-producing B lymphocytes from the immunized animal are then fused with cells of an immortal myeloma cell, generally one of the same species as the animal that was immunized or human or human/mouse chimeric cells. Myeloma cell lines suited for use in hybridoma-producing fusion procedures preferably are non-antibody-producing, have high fusion efficiency, and enzyme deficiencies that render then incapable of growing in certain selective media which support the growth of only the desired fused cells (hybridomas).

Any one of a number of myeloma cells may be used, as are known to those of skill in the art (Goding, pp. 65-66, 1986; Campbell, pp. 75-83, 1984). For example, where the immunized animal is a mouse, one may use P3-X63/Ag8, X63-Ag8.653, NS1/1.Ag 4 1, Sp210-Ag14, FO, NSO/U, MPC-11, MPC11-X45-GTG 1.7 and S194/5XX0 Bul; for rats, one may use R210.RCY3, Y3-Ag 1.2.3, IR983F and 4B210; and U-266, GM1500-GRG2, LICR-LON-HMy2 and UC729-6 are all useful in connection with human cell fusions. One particular murine myeloma cell is the NS-1 myeloma cell line (also termed P3-NS-1-Ag4-1), which is readily available from the NIGMS Human Genetic Mutant Cell Repository by requesting cell line repository number GM3573. Another mouse myeloma cell line that may be used is the 8-azaguanine-resistant mouse murine myeloma SP2/0 non-producer cell line. More recently, additional fusion partner lines for use with human B cells have been described, including KR12 (ATCC CRL-8658; K6H6/B5 (ATCC CRL-1823 SHM-D33 (ATCC CRL-1668) and HMMA2.5 (Posner et al., 1987). The antibodies in this disclosure were generated using the SP2/0/mIL-6 cell line, an IL-6 secreting derivative of the SP2/0 line.

Methods for generating hybrids of antibody-producing spleen or lymph node cells and myeloma cells usually comprise mixing somatic cells with myeloma cells in a 2:1 proportion, though the proportion may vary from about 20:1 to about 1:1, respectively, in the presence of an agent or agents (chemical or electrical) that promote the fusion of cell membranes. Fusion methods using Sendai virus have been described by Kohler and Milstein (1975; 1976), and those using polyethylene glycol (PEG), such as 37% (v/v) PEG, by Gefter et al. (1977). The use of electrically induced fusion methods also is appropriate (Goding, pp. 71-74, 1986).
Fusion procedures usually produce viable hybrids at low frequencies, about 1×10⁻⁶ to 1×10⁻⁸. However, this does not pose a problem, as the viable, fused hybrids are differentiated from the parental, infused cells (particularly the infused myeloma cells that would normally continue to divide indefinitely) by culturing in a selective medium. The selective medium is generally one that contains an agent that blocks the de novo synthesis of nucleotides in the tissue culture media. Exemplary and preferred agents are aminopterin, methotrexate, and azaserine. Aminopterin and methotrexate block de novo synthesis of both purines and pyrimidines, whereas azaserine blocks only purine synthesis. Where aminopterin or methotrexate is used, the media is supplemented with hypoxanthine and thymidine as a source of nucleotides (HAT medium). Where azaserine is used, the media is supplemented with hypoxanthine. Ouabain is added if the B cell source is an Epstein Barr virus (EBV) transformed human B cell line, in order to eliminate EBV transformed lines that have not fused to the myeloma.
The preferred selection medium is HAT or HAT with ouabain. Only cells capable of operating nucleotide salvage pathways are able to survive in HAT medium. The myeloma cells are defective in key enzymes of the salvage pathway, e.g., hypoxanthine phosphoribosyl transferase (HPRT), and they cannot survive. The B cells can operate this pathway, but they have a limited life span in culture and generally die within about two weeks. Therefore, the only cells that can survive in the selective media are those hybrids formed from myeloma and B cells. When the source of B cells used for fusion is a line of EBV-transformed B cells, as here, ouabain is also used for drug selection of hybrids as EBV-transformed B cells are susceptible to drug killing, whereas the myeloma partner used is chosen to be ouabain resistant.
Culturing provides a population of hybridomas from which specific hybridomas are selected. Typically, selection of hybridomas is performed by culturing the cells by single-clone dilution in microtiter plates, followed by testing the individual clonal supernatants (after about two to three weeks) for the desired reactivity. The assay should be sensitive, simple and rapid, such as radioimmunoassays, enzyme immunoassays, cytotoxicity assays, plaque assays dot immunobinding assays, and the like.
The selected hybridomas are then serially diluted or single-cell sorted by flow cytometric sorting and cloned into individual antibody-producing cell lines, which clones can then be propagated indefinitely to provide mAbs. The cell lines may be exploited for MAb production in two basic ways. A sample of the hybridoma can be injected (often into the peritoneal cavity) into an animal (e.g., a mouse). Optionally, the animals are primed with a hydrocarbon, especially oils such as pristane (tetramethylpentadecane) prior to injection. When human hybridomas are used in this way, it is optimal to inject immunocompromised mice, such as SCID mice, to prevent tumor rejection. The injected animal develops tumors secreting the specific monoclonal antibody produced by the fused cell hybrid. The body fluids of the animal, such as serum or ascites fluid, can then be tapped to provide MAbs in high concentration. The individual cell lines could also be cultured in vitro, where the MAbs are naturally secreted into the culture medium from which they can be readily obtained in high concentrations. Alternatively, human hybridoma cells lines can be used in vitro to produce immunoglobulins in cell supernatant. The cell lines can be adapted for growth in serum-free medium to optimize the ability to recover human monoclonal immunoglobulins of high purity.
Fragments of the monoclonal antibodies of the disclosure can be obtained from the purified monoclonal antibodies by methods which include digestion with enzymes, such as pepsin or papain, and/or by cleavage of disulfide bonds by chemical reduction. Alternatively, monoclonal antibody fragments encompassed by the present disclosure can be synthesized using an automated peptide synthesizer.
It also is contemplated that a molecular cloning approach may be used to generate monoclonals. For this, RNA can be isolated from the hybridoma line and the antibody genes obtained by RT-PCR and cloned into an immunoglobulin expression vector. Alternatively, combinatorial immunoglobulin phagemid libraries are prepared from RNA isolated from the cell lines and phagemids expressing appropriate antibodies are selected by panning using viral antigens. The advantages of this approach over conventional hybridoma techniques are that approximately 10 (Berglund et al., 2009) times as many antibodies can be produced and screened in a single round, and that new specificities are generated by H and L chain combination which further increases the chance of finding appropriate antibodies.
Other U.S. patents, each incorporated herein by reference, that teach the production of antibodies useful in the present disclosure include U.S. Pat. No. 5,565,332, which describes the production of chimeric antibodies using a combinatorial approach; U.S. Pat. No. 4,816,567 which describes recombinant immunoglobulin preparations; and U.S. Pat. No. 4,867,973 which describes antibody-therapeutic agent conjugates.

### B. Engineering of Antibody Sequences

In various embodiments, one may choose to engineer sequences of the identified antibodies for a variety of reasons, such as improved expression, improved cross-reactivity, diminished off-target binding or abrogation of one or more natural effector functions, such as activation of complement or recruitment of immune cells (e.g., T cells). In particular, IgM antibodies may be converted to IgG antibodies. The following is a general discussion of relevant techniques for antibody engineering.

Hybridomas may be cultured, then cells lysed, and total RNA extracted. Random hexamers may be used with RT to generate cDNA copies of RNA, and then PCR performed using a multiplex mixture of PCR primers expected to amplify all human variable gene sequences. PCR product can be cloned into pGEM-T Easy vector, then sequenced by automated DNA sequencing using standard vector primers. Assay of binding and neutralization may be performed using antibodies collected from hybridoma supernatants and purified by FPLC, using Protein G columns. Recombinant full length IgG antibodies can be generated by subcloning heavy and light chain Fv DNAs from the cloning vector into a Lonza pConIgG1 or pConK2 plasmid vector, transfected into 293 Freestyle cells or Lonza CHO cells, and collected and purified from the CHO cell supernatant.

The rapid availability of antibody produced in the same host cell and cell culture process as the final cGMP manufacturing process has the potential to reduce the duration of process development programs. Lonza has developed a generic method using pooled transfectants grown in CDACF medium, for the rapid production of small quantities (up to 50 g) of antibodies in CHO cells. Although slightly slower than a true transient system, the advantages include a higher product concentration and use of the same host and process as the production cell line. Example of growth and productivity of GS-CHO pools, expressing a model antibody, in a disposable bioreactor: in a disposable bag bioreactor culture (5 L working volume) operated in fed-batch mode, a harvest antibody concentration of 2 g/L was achieved within 9 weeks of transfection.

pCon Vectors™ are an easy way to re-express whole antibodies. The constant region vectors are a set of vectors offering a range of immunoglobulin constant region vectors cloned into the pEE vectors. These vectors offer easy construction of full length antibodies with human constant regions and the convenience of the GS System™.

Antibody molecules will comprise fragments (such as F(ab′), F(ab′)₂) that are produced, for example, by the proteolytic cleavage of the mAbs, or single-chain immunoglobulins producible, for example, via recombinant means. Such antibody derivatives are monovalent. In one embodiment, such fragments can be combined with one another, or with other antibody fragments or receptor ligands to form “chimeric” binding molecules. Significantly, such chimeric molecules may contain substituents capable of binding to different epitopes of the same molecule.
It may be desirable to “humanize” antibodies produced in non-human hosts in order to attenuate any immune reaction when used in human therapy. Such humanized antibodies may be studied in an in vitro or an in vivo context. Humanized antibodies may be produced, for example by replacing an immunogenic portion of an antibody with a corresponding, but non-immunogenic portion (i.e., chimeric antibodies). PCT Application PCT/US86/02269; EP Application 184,187; EP Application 171,496; EP Application 173,494; PCT Application WO 86/01533; EP Application 125,023; Sun et al. (1987); Wood et al. (1985); and Shaw et al. (1988); all of which references are incorporated herein by reference. General reviews of “humanized” chimeric antibodies are provided by Morrison (1985); also incorporated herein by reference. “Humanized” antibodies can alternatively be produced by CDR or CEA substitution. Jones et al. (1986); Verhoeyen et al. (1988); Beidler et al. (1988); all of which are incorporated herein by reference.

In related embodiments, the antibody is a derivative of the disclosed antibodies, e.g., an antibody comprising the CDR sequences identical to those in the disclosed antibodies (e.g., a chimeric, humanized or CDR-grafted antibody). In yet a further embodiment, the antibody is a fully human recombinant antibody.

The present disclosure also contemplates isotype modification. By modifying the Fc region to have a different isotype, different functionalities can be achieved. For example, changing to IgG₄ can reduce immune effector functions associated with other isotypes.

Modified antibodies may be made by any technique known to those of skill in the art, including expression through standard molecular biological techniques, or the chemical synthesis of polypeptides. Methods for recombinant expression are addressed elsewhere in this document.

### C. Expression

Nucleic acids according to the present disclosure will encode antibodies, optionally linked to other protein sequences. As used in this application, the term “a nucleic acid encoding an IGFBP1 antibody” refers to a nucleic acid molecule that has been isolated free of total cellular nucleic acid.

The DNA segments of the present disclosure include those encoding biologically functional equivalent proteins and peptides of the sequences described above. Such sequences may arise as a consequence of codon redundancy and amino acid functional equivalency that are known to occur naturally within nucleic acid sequences and the proteins thus encoded. Alternatively, functionally equivalent proteins or peptides may be created via the application of recombinant DNA technology, in which changes in the protein structure may be engineered, based on considerations of the properties of the amino acids being exchanged. Changes designed by man may be introduced through the application of site-directed mutagenesis techniques or may be introduced randomly and screened later for the desired function, as described below.

Within certain embodiments, expression vectors are employed to express an antibody. In other embodiments, the expression vectors are used in gene therapy. Expression requires that appropriate signals be provided in the vectors, and which include various regulatory elements, such as enhancers/promoters from both viral and mammalian sources that drive expression of the genes of interest in host cells. Elements designed to optimize messenger RNA stability and translatability in host cells also are defined. The conditions for the use of a number of dominant drug selection markers for establishing permanent, stable cell clones expressing the products are also provided, as is an element that links expression of the drug selection markers to expression of the polypeptide.

Throughout this application, the term “expression construct” is meant to include any type of genetic construct containing a nucleic acid coding for a gene product in which part or all of the nucleic acid encoding sequence is capable of being transcribed. The transcript may be translated into a protein, but it need not be. In certain embodiments, expression includes both transcription of a gene and translation of mRNA into a gene product. In other embodiments, expression only includes transcription of the nucleic acid encoding a gene of interest.

The term “vector” is used to refer to a carrier nucleic acid molecule into which a nucleic acid sequence can be inserted for introduction into a cell where it can be replicated. A nucleic acid sequence can be “exogenous,” which means that it is foreign to the cell into which the vector is being introduced or that the sequence is homologous to a sequence in the cell but in a position within the host cell nucleic acid in which the sequence is ordinarily not found. Vectors include plasmids, cosmids, viruses (bacteriophage, animal viruses, and plant viruses), and artificial chromosomes (e.g., YACs). One of skill in the art would be well equipped to construct a vector through standard recombinant techniques, which are described in Sambrook et al. (1989) and Ausubel et al. (1994), both incorporated herein by reference.

The term “expression vector” refers to a vector containing a nucleic acid sequence coding for at least part of a gene product capable of being transcribed. In some cases, RNA molecules are then translated into a protein, polypeptide, or peptide. In other cases, these sequences are not translated, for example, in the production of antisense molecules or ribozymes. Expression vectors can contain a variety of “control sequences,” which refer to nucleic acid sequences necessary for the transcription and possibly translation of an operably linked coding sequence in a particular host organism, often referred to as promoters. In addition to control sequences that govern transcription and translation, vectors and expression vectors may contain nucleic acid sequences that serve other functions as well.

Numerous expression systems exist that comprise at least a part or all of the compositions discussed above. Prokaryote- and/or eukaryote-based systems can be employed for use with the present disclosure to produce nucleic acid sequences, or their cognate polypeptides, proteins and peptides. Many such systems are commercially and widely available.

The insect cell/baculovirus system can produce a high level of protein expression of a heterologous nucleic acid segment, such as described in U.S. Pat. Nos. 5,871,986 and 4,879,236, both herein incorporated by reference, and which can be bought, for example, under the name MaxBac® 2.0 from Invitrogen® and BacPack™ Baculovirus Expression System From Clontech®.

Other examples of expression systems include Stratagene®'s Complete Control™ Inducible Mammalian Expression System, which involves a synthetic ecdysone-inducible receptor, or its pET Expression System, an E. coli expression system. Another example of an inducible expression system is available from Invitrogen®, which carries the T-Rex™ (tetracycline-regulated expression) System, an inducible mammalian expression system that uses the full-length CMV promoter. Invitrogen® also provides a yeast expression system called the Pichia methanolica Expression System, which is designed for high-level production of recombinant proteins in the methylotrophic yeast Pichia methanolica. One of skill in the art would know how to express a vector, such as an expression construct, to produce a nucleic acid sequence or its cognate polypeptide, protein, or peptide.

Primary mammalian cell cultures may be prepared in various ways. In order for the cells to be kept viable while in vitro and in contact with the expression construct, it is necessary to ensure that the cells maintain contact with the correct ratio of oxygen and carbon dioxide and nutrients but are protected from microbial contamination. Cell culture techniques are well documented.

One embodiment of the foregoing involves the use of gene transfer to immortalize cells for the production of proteins. The gene for the protein of interest may be transferred as described above into appropriate host cells followed by culture of cells under the appropriate conditions. The gene for virtually any polypeptide may be employed in this manner. The generation of recombinant expression vectors, and the elements included therein, are discussed above. Alternatively, the protein to be produced may be an endogenous protein normally synthesized by the cell in question.

Examples of useful mammalian host cell lines are Vero and HeLa cells and cell lines of Chinese hamster ovary, W138, BHK, COS-7, 293, HepG2, NIH3T3, RIN and MDCK cells. In addition, a host cell strain may be chosen that modulates the expression of the inserted sequences, or modifies and process the gene product in the manner desired. Such modifications (e.g., glycosylation) and processing (e.g., cleavage) of protein products may be important for the function of the protein. Different host cells have characteristic and specific mechanisms for the post-translational processing and modification of proteins. Appropriate cell lines or host systems can be chosen to insure the correct modification and processing of the foreign protein expressed.

### D. Purification

In certain embodiments, the antibodies of the present disclosure may be purified. The term “purified,” as used herein, is intended to refer to a composition, isolatable from other components, wherein the protein is purified to any degree relative to its naturally-obtainable state. A purified protein therefore also refers to a protein, free from the environment in which it may naturally occur. Where the term “substantially purified” is used, this designation will refer to a composition in which the protein or peptide forms the major component of the composition, such as constituting about 50%, about 60%, about 70%, about 80%, about 90%, about 95% or more of the proteins in the composition.
Protein purification techniques are well known to those of skill in the art, such as filtration, centrifugation and various chromatographic methods such as FPLC or affinity chromatography. All of these techniques involve, at one level, the crude fractionation of the cellular milieu to polypeptide and non-polypeptide fractions. Having separated the polypeptide from other proteins, the polypeptide of interest may be further purified using chromatographic and electrophoretic techniques to achieve partial or complete purification (or purification to homogeneity). Analytical methods particularly suited to the preparation of a pure peptide are ion-exchange chromatography, exclusion chromatography; polyacrylamide gel electrophoresis; isoelectric focusing. Other methods for protein purification include, precipitation with ammonium sulfate, PEG, antibodies and the like or by heat denaturation, followed by centrifugation; gel filtration, reverse phase, hydroxylapatite and affinity chromatography; and combinations of such and other techniques.
In purifying an antibody of the present disclosure, it may be desirable to express the polypeptide in a prokaryotic or eukaryotic expression system and extract the protein using denaturing conditions. The polypeptide may be purified from other cellular components using an affinity column, which binds to a tagged portion of the polypeptide. As is generally known in the art, it is believed that the order of conducting the various purification steps may be changed, or that certain steps may be omitted, and still result in a suitable method for the preparation of a substantially purified protein or peptide.
Commonly, complete antibodies are fractionated utilizing agents (i.e., protein A) that bind the Fc portion of the antibody. Alternatively, antigens may be used to simultaneously purify and select appropriate antibodies. Such methods often utilize the selection agent bound to a support, such as a column, filter or bead. The antibodies are bound to a support, contaminants removed (e.g., washed away), and the antibodies released by applying conditions (salt, heat, etc.).
Various methods for quantifying the degree of purification of the protein or peptide will be known to those of skill in the art in light of the present disclosure. These include, for example, determining the specific activity of an active fraction, or assessing the amount of polypeptides within a fraction by SDS/PAGE analysis. Another method for assessing the purity of a fraction is to calculate the specific activity of the fraction, to compare it to the specific activity of the initial extract, and to thus calculate the degree of purity. The actual units used to represent the amount of activity will, of course, be dependent upon the particular assay technique chosen to follow the purification and whether or not the expressed protein or peptide exhibits a detectable activity.

It is known that the migration of a polypeptide can vary, sometimes significantly, with different conditions of SDS/PAGE (Capaldi et al., 1977). It will therefore be appreciated that under differing electrophoresis conditions, the apparent molecular weights of purified or partially purified expression products may vary.

### E. Single Chain/Single Domain Antibodies

A Single Chain Variable Fragment (scFv) is a fusion of the variable regions of the heavy and light chains of immunoglobulins, linked together with a short (usually serine, glycine) linker. This chimeric molecule, also known as a single domain antibody, retains the specificity of the original immunoglobulin, despite removal of the constant regions and the introduction of a linker peptide. This modification usually leaves the specificity unaltered. These molecules were created historically to facilitate phage display where it is highly convenient to express the antigen binding domain as a single peptide. Alternatively, scFv can be created directly from subcloned heavy and light chains derived from a hybridoma. Single domain or single chain variable fragments lack the constant Fc region found in complete antibody molecules, and thus, the common binding sites (e.g., protein A/G) used to purify antibodies (single chain antibodies include the Fc region). These fragments can often be purified/immobilized using Protein L, since Protein L interacts with the variable region of kappa light chains.

Flexible linkers generally are comprised of helix- and turn-promoting amino acid residues such as alaine, serine and glycine. However, other residues can function as well. Tang et al. (1996) used phage display as a means of rapidly selecting tailored linkers for single-chain antibodies (scFvs) from protein linker libraries. A random linker library was constructed in which the genes for the heavy and light chain variable domains were linked by a segment encoding an 18-amino acid polypeptide of variable composition. The scFv repertoire (approx. 5×10 (Kharitonenkov et al., 2005) different members) was displayed on filamentous phage and subjected to affinity selection with hapten. The population of selected variants exhibited significant increases in binding activity but retained considerable sequence diversity. Screening 1054 individual variants subsequently yielded a catalytically active scFv that was produced efficiently in soluble form. Sequence analysis revealed a conserved proline in the linker two residues after the V_{H} C terminus and an abundance of arginines and prolines at other positions as the only common features of the selected tethers.

The recombinant antibodies of the present disclosure may also involve sequences or moieties that permit dimerization or multimerization of the receptors. Such sequences include those derived from IgA, which permit formation of multimers in conjunction with the J-chain. Another multimerization domain is the Gal4 dimerization domain. In other embodiments, the chains may be modified with agents such as biotin/avidin, which permit the combination of two antibodies.

In a separate embodiment, a single-chain antibody can be created by joining receptor light and heavy chains using a non-peptide linker or chemical unit. Generally, the light and heavy chains will be produced in distinct cells, purified, and subsequently linked together in an appropriate fashion (i.e., the N-terminus of the heavy chain being attached to the C-terminus of the light chain via an appropriate chemical bridge).

Cross-linking reagents are used to form molecular bridges that tie functional groups of two different molecules, e.g., a stablizing and coagulating agent. However, it is contemplated that dimers or multimers of the same analog or heteromeric complexes comprised of different analogs can be created. To link two different compounds in a step-wise manner, hetero-bifunctional cross-linkers can be used that eliminate unwanted homopolymer formation.
An exemplary hetero-bifunctional cross-linker contains two reactive groups: one reacting with primary amine group (e.g., N-hydroxy succinimide) and the other reacting with a thiol group (e.g., pyridyl disulfide, maleimides, halogens, etc.). Through the primary amine reactive group, the cross-linker may react with the lysine residue(s) of one protein (e.g., the selected antibody or fragment) and through the thiol reactive group, the cross-linker, already tied up to the first protein, reacts with the cysteine residue (free sulfhydryl group) of the other protein (e.g., the selective agent).
It is preferred that a cross-linker having reasonable stability in blood will be employed. Numerous types of disulfide-bond containing linkers are known that can be successfully employed to conjugate targeting and therapeutic/preventative agents. Linkers that contain a disulfide bond that is sterically hindered may prove to give greater stability in vivo, preventing release of the targeting peptide prior to reaching the site of action. These linkers are thus one group of linking agents.
Another cross-linking reagent is SMPT, which is a bifunctional cross-linker containing a disulfide bond that is “sterically hindered” by an adjacent benzene ring and methyl groups. It is believed that steric hindrance of the disulfide bond serves a function of protecting the bond from attack by thiolate anions such as glutathione which can be present in tissues and blood, and thereby help in preventing decoupling of the conjugate prior to the delivery of the attached agent to the target site.
The SMPT cross-linking reagent, as with many other known cross-linking reagents, lends the ability to cross-link functional groups such as the SH of cysteine or primary amines (e.g., the epsilon amino group of lysine). Another possible type of cross-linker includes the hetero-bifunctional photoreactive phenylazides containing a cleavable disulfide bond such as sulfosuccinimidyl-2-(p-azido salicylamido) ethyl-1,3′-dithiopropionate. The N-hydroxy-succinimidyl group reacts with primary amino groups and the phenylazide (upon photolysis) reacts non-selectively with any amino acid residue.
In addition to hindered cross-linkers, non-hindered linkers also can be employed in accordance herewith. Other useful cross-linkers, not considered to contain or generate a protected disulfide, include SATA, SPDP and 2-iminothiolane (Wawrzynczak & Thorpe, 1987). The use of such cross-linkers is well understood in the art. Another embodiment involves the use of flexible linkers.
U.S. Pat. No. 4,680,338, describes bifunctional linkers useful for producing conjugates of ligands with amine-containing polymers and/or proteins, especially for forming antibody conjugates with chelators, drugs, enzymes, detectable labels and the like. U.S. Pat. Nos. 5,141,648 and 5,563,250 disclose cleavable conjugates containing a labile bond that is cleavable under a variety of mild conditions. This linker is particularly useful in that the agent of interest may be bonded directly to the linker, with cleavage resulting in release of the active agent. Particular uses include adding a free amino or free sulfhydryl group to a protein, such as an antibody, or a drug.
U.S. Pat. No. 5,856,456 provides peptide linkers for use in connecting polypeptide constituents to make fusion proteins, e.g., single chain antibodies. The linker is up to about 50 amino acids in length, contains at least one occurrence of a charged amino acid (preferably arginine or lysine) followed by a proline, and is characterized by greater stability and reduced aggregation. U.S. Pat. No. 5,880,270 discloses aminooxy-containing linkers useful in a variety of immunodiagnostic and separative techniques.

### F. Antibody Conjugates

Antibodies may be linked to at least one agent to form an antibody conjugate. In order to increase the efficacy of antibody molecules as diagnostic or therapeutic agents, it is conventional to link or covalently bind or complex at least one desired molecule or moiety. Such a molecule or moiety may be, but is not limited to, at least one effector or reporter molecule. Effector molecules comprise molecules having a desired activity, e.g., immunosuppression/anti-inflammation. Non-limiting examples of such molecules are set out above. Such molecules are optionally attached via cleavable linkers designed to allow the molecules to be released at or near the target site.

By contrast, a reporter molecule is defined as any moiety which may be detected using an assay. Non-limiting examples of reporter molecules which have been conjugated to antibodies include enzymes, radiolabels, haptens, fluorescent labels, phosphorescent molecules, chemiluminescent molecules, chromophores, photoaffinity molecules, colored particles or ligands, such as biotin.

### G. Commercially Available Antibodies

There are a number of commercially available anti-IGFBP1 antibodies. M-19 is a goat polyclonal IgG and is available from Santa Cruz Biotech. ab10732 and ab 111203, both available from Abcam, are mouse and rabbit polyclonals, respectively. Pierce Antibodies sells a monoclonal antibody (MA1-24775) as does abnova (clone 2F9 and B787M) and Santa Cruz Biotech (H-5). Numerous others are available.

### V. Methods of Treatment

A. Menopause-Induced Bone Loss

Menopause is the cessation of a woman's reproductive ability, the opposite of menarche. Menopause is usually a natural change; it typically occurs in women in midlife, during their late 40's or early 50's, signaling the end of the fertile phase of a woman's life. Menopause is commonly defined by the state of the uterus and the absence of menstrual flow, but it can instead be more accurately defined as the permanent cessation of the primary functions of the ovaries. What ceases is the ripening and release of ova and the release of hormones that cause both the build-up of the uterine lining, and the subsequent shedding of the uterine lining (the menses or period).

The transition from a potentially reproductive to a non-reproductive state is normally not sudden or abrupt, occurs over a number of years, and is a consequence of biological aging. For some women, during the transition years the accompanying signs and effects (including lack of energy, hot flashes, and mood changes) can be powerful enough to significantly disrupt their daily activities and sense of well-being. In those cases various different treatments can be tried.

Medically speaking, the date of menopause (in a woman with an intact uterus) is the day after the final episode of menstrual flow finishes. “Perimenopause” is a term for the menopause transition years, the time both before and after the last period ever, while hormone levels are still fluctuating erratically. “Premenopause” is a term for the years leading up to menopause. “Postmenopause” is the part of a woman's life that occurs after the date of menopause; once a woman with an intact uterus (who is not pregnant or lactating) has gone a year with no flow at all she is considered to be one year into post menopause.

During the menopause transition years, as the body responds to the rapidly fluctuating and dropping levels of the body's own hormones, a number of effects may appear. Not every woman experiences bothersome levels of these effects; the degree to which they occur varies greatly from person to person.

The majority of women find that their menstrual periods are gradually becoming more erratic, and the timing of the start of the flow usually becomes more and more difficult to predict. In addition the duration of the flow may be considerably shorter or longer than normal, and the flow itself may be significantly heavier or lighter than was previously the case, including sometimes long episodes of spotting.

It is not uncommon to have a 2-week cycle when an ovulation has been skipped. Further into the process it is common to skip periods for months at a time, and these skipped periods may be followed by a heavier period. The number of skipped periods in a row often increases as the time of last period approaches. If a woman keeps a written record of all the erratic episodes of flow, she will know how many months have passed with no flow at all, and thus will be able to know at what date she reached postmenopause, which is important medical information that will subsequently frequently be requested by doctors.

Effects such as formication (crawling, itching, or tingling skin sensations), may be associated directly with hormone withdrawal. Effects that are caused by the extreme fluctuations in hormone levels (for example hot flashes and mood changes) will usually disappear or improve significantly once the perimenopause transition is completely over, however, effects that are due to low estrogen levels (for example vaginal atrophy and skin drying) will continue after the menopause transition years are over.

Hot flashes and mood changes are the most commonly mentioned symptoms of perimenopause, but in a 2007 study, lack of energy was identified by women as the most distressing effect. Other effects can include palpitations, psychological effects such as depression, anxiety, irritability, memory problems and lack of concentration, and atrophic effects such as vaginal dryness and urgency of urination.

Another major symptom of menopause is osteoporosis. Osteoporosis is a progressive bone disease that is characterized by a decrease in bone mass and density which can lead to an increased risk of fracture. In osteoporosis, the bone mineral density (BMD) is reduced, bone microarchitecture deteriorates, and the amount and variety of proteins in bone are altered. Osteoporosis is defined by the World Health Organization (WHO) as a bone mineral density of 2.5 standard deviations or more below the mean peak bone mass (average of young, healthy adults) as measured by dual-energy X-ray absorptiometry; the term “established osteoporosis” includes the presence of a fragility fracture. The disease may be classified as primary type 1, primary type 2, or secondary. The form of osteoporosis most common in women after menopause is referred to as primary type 1 or postmenopausal osteoporosis. Primary type 2 osteoporosis or senile osteoporosis occurs after age 75 and is seen in both females and males at a ratio of 2:1. Secondary osteoporosis may arise at any age and affect men and women equally. This form results from chronic predisposing medical problems or disease, or prolonged use of medications such as glucocorticoids, when the disease is called steroid- or glucocorticoid-induced osteoporosis.

The risk of osteoporosis fractures can be reduced with lifestyle changes and in those with previous osteoporosis related fractures, medications. Lifestyle change includes diet, exercise, and preventing falls. The utility of calcium and vitamin D is questionable in most. Bisphosphonates are useful in those with previous fractures from osteoporosis but are of minimal benefit in those who have osteoporosis but no previous fractures. Osteoporosis is a component of the frailty syndrome.

B. FGF21-Induced Bone Resorption

Type-2 diabetes is a pathological condition in which glucose levels are higher than normal in the blood. Usually, sufferers of type 2 diabetes cannot properly use insulin, produced by their pancreas, to degrade or store the excess of glucose after each meal. This is called insulin intolerance and is a common consequence of metabolic disorders mediated by obesity. More precisely, obesity and bad metabolism cause the increase of lipids, such as cholesterol and triglycerides.

Current treatment for type-2 diabetes involves a combinatorial strategy and the prescription of several medications as no single drug can treat multiple metabolic markers at the same time. This makes it difficult for many patients to continue therapy. It also causes intolerance leading to ineffective therapy. Scientists have previously discovered that FGF21 plays an important role in lipid and energy metabolism. When diabetic mice were given FGF21 protein, an improvement in all metabolic symptoms was observed. Currently there are a number of FGF21 analogs in development.

However, the inventor has shown that FGF21 is a negative regulator of bone. Mechanistically, FGF21 forms a feed-forward loop to mediate and enhance PPAR-γactivity, thereby potentiating the ability of PPAR-γ agonist to inhibit osteoblastogenesis and stimulate adipogenesis from bone marrow MSCs. Consequently, FGF21 deletion confers resistance to rosiglitazone-induced bone loss. Importantly, these results suggest that, despite the beneficial effects of FGF21 in treating insulin resistance and type 2 diabetes, long-term FGF21 administration may cause skeletal fragility. Thus, the ability to impede the bone-destructive aspect of FGF21 without impacting its benefits to obese and/or diabetic patients is highly significant.

C. Pharmaceutical Formulations

The present disclosure provides pharmaceutical compositions comprising anti-IGFBP1 antibodies. In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Other suitable pharmaceutical excipients include starch, glucose, lactose, sucrose, saline, dextrose, gelatin, malt, rice, flour, chalk, silica gel, sodium stearate, glycerol monostearate, talc, sodium chloride, dried skim milk, glycerol, propylene glycol, water, ethanol and the like.

The compositions can be formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

The antibodies of the present disclosure may include classic pharmaceutical preparations. Administration of these compositions according to the present disclosure will be via any common route so long as the target tissue is available via that route. This includes oral, nasal, buccal, rectal, vaginal or topical. Alternatively, administration may be by intradermal, subcutaneous, intramuscular, intraperitoneal or intravenous injection. Such compositions would normally be administered as pharmaceutically acceptable compositions, described supra. Of particular interest is administration local or regional to a site of bone loss.
The antibodies may also be administered parenterally or intraperitoneally. Solutions of the active compounds as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

D. Combination Therapies

In the context of the present disclosure, it also is contemplated that anti-IGFBP1 antibodies described herein could be used similarly in conjunction with other treatments to reduce bone loss/resorption. It also may prove effective, in particular, to combine anti-IGFBP1 antibodies with other therapies that target different aspects of bone formation/loss.
These compositions are be provided in a combined amount effective to treat a disease or condition. This process may involve administering the anti-IGFBP1 antibody according to the present disclosure and the other agent(s) or factor(s) at the same time. This may be achieved by use of a single composition or pharmacological formulation that includes both agents, or with two distinct compositions or formulations, at the same time, wherein one composition includes the anti-IGFBP1 antibody according to the present disclosure and the other includes the other agent.
Alternatively, the anti-IGFBP1 antibody therapy may precede or follow the other agent treatment by intervals ranging from minutes to weeks. In embodiments where the other agent and the anti-IGFBP1 antibody are administered separately, one would generally ensure that a significant period of time did not expire between the time of each delivery, such that the agents would still be able to exert an advantageously combined effect on the subject. In such instances, it is contemplated that one would provide both modalities within about 12-24 hours of each other and, more preferably, within about 6-12 hours of each other, with a delay time of only about 12 hours being most preferred. In some situations, it may be desirable to extend the time period for treatment significantly, however, where several days (2, 3, 4, 5, 6 or 7) to several weeks (1, 2, 3, 4, 5, 6, 7 or 8) lapse between the respective administrations. It also is conceivable that more than one administration of either anti-IGFBP1 antibody or the other agent will be desired. Various combinations may be employed, where an anti-IGFBP1 antibody according to the present disclosure is “A” and the other therapy is “B”, as exemplified below:

A/B/A B/A/B B/B/A A/A/B B/A/A A/B/B B/B/B/A B/B/A/B
A/A/B/B A/B/A/B A/B/B/A B/B/A/A B/A/B/A B/A/A/B B/B/B/A
A/A/A/B B/A/A/A A/B/A/A A/A/B/A A/B/B/B B/A/B/B B/B/A/B

Other combinations are contemplated.

### VI. Examples

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Materials and Methods

### Mice.

FGF21-Tg and β-Klotho flox mice have been described (Ding et al., 2012 and Inagaki et al., 2007). Liver-specific β-Klotho knockout mice were generated by crossing β-Klotho flox mice with albumin-cre mice (Jackson Laboratory). Fat- and brain-specific β-Klotho knockout mice were previously described (Ding et al., 2012 and Bookout et al., 2013). To generate conditional integrin β1 knockout mice in the macrophage/osteoclast lineage, the inventor bred Integrin β1 flox mice (Raghavan et al., 2000) with lysozyme-cre mice (Clausen et al., 1999) (Jackson laboratory). Ovariectomy or sham operation was performed on 10-20 week old female mice as previously described (Wei et al., 2010). Insulin tolerance test (ITT) was performed as previously described (Ding et al., 2012). Mice were fed standard chow containing 4% fat ad libitum unless stated otherwise. For diet-induced-obesity, mice were fed a high fat diet containing 60% kcal from fat (Research Diets Inc. #D12492). All experiments were conducted using littermates and all mice were on C57B6/J background. All animal experiments were approved by the Institutional Animal Care and Use Committee of the University of Texas Southwestern Medical Center.

### Reagent.

Recombinant mouse and human IGFBP1, recombinant mouse MCSF, recombinant mouse and human RANKL and anti-IGFBP1 antibody were from R&D Systems. RGD-containing peptide (Gly-Arg-Gly-Asp-Thr-Pro) was from Sigma. Recombinant human FGF21 (residues 29-209) was expressed and purified as described (Goetz et al., 2007). Antibodies for p-ERK and total ERK were from Cell Signaling.

### Bone Analyses.

Micro-Computed Tomography (μCT) was performed to evaluate bone volume and architecture using a Scanco μCT-35 instrument (SCANCO Medical) as described (Wei et al., 2012 and Wei et al., 2010). As a bone resorption marker, serum CTX-1 was measured with the RatLaps™ EIA kit (Immunodiagnostic Systems) (Wei et al., 2012). As a bone formation marker, serum PINP was measured with the Rat/Mouse PINP EIA kit (Immunodiagnostic Systems) (Wei et al., 2012).

### Bone Marrow Osteoclast Differentiation.

Osteoclasts were differentiated from mouse bone marrow cells as described (Wan et al., 2007 and Wei et al., 2010). Briefly, hematopoietic bone marrow cells were purified with 40 μm cell strainer, and differentiated with 40 ng/ml of mouse M-CSF (R&D Systems) in α-MEM containing 10% FBS for 3 days, then with 40 ng/ml of mouse MCSF and 100 ng/ml of mouse RANKL (R&D Systems) for 3-9 days, in the presence or absence of rosiglitazone (1 μM). Mature osteoclasts were identified as multinucleated (>3 nuclei) TRAP⁺ cells. Osteoclast differentiation was quantified by the RNA expression of osteoclast marker genes using RT-QPCR analysis. To determine the effects of liver-secreted factors on osteoclast differentiation, fresh liver was minced in culture medium (α-MEM+10% FBS) and dispersed by gentle vortex for 5 min; cells were pelleted by centrifugation at 10K rpm for 10 min; supernatant was collected as liver conditioned medium (LCM) and used for osteoclast differentiation culture. Human PBMN cells were differentiated into osteoclasts in α-MEM containing 10% FBS, 25 ng/ml MCSF, 50 ng/ml hRANKL, 1 μM Dexamethasome and 1 μM rosiglitazone for 14 days, in the presence of 500 ng/ml human IGFBP1 or PBS control.

### Statistical Analyses.

All statistical analyses were performed with Student's t-Test and presented as mean±standard deviation (s.d.) unless stated otherwise. The p values were designated as: *, p<0.05; **, p<0.01; ***, p<0.005; ****, p<0.001; n.s. non-significant (p>0.05).

### Example 2

### Results and Discussion

### Liver β-Klotho Deletion Reduces Bone Resorption.

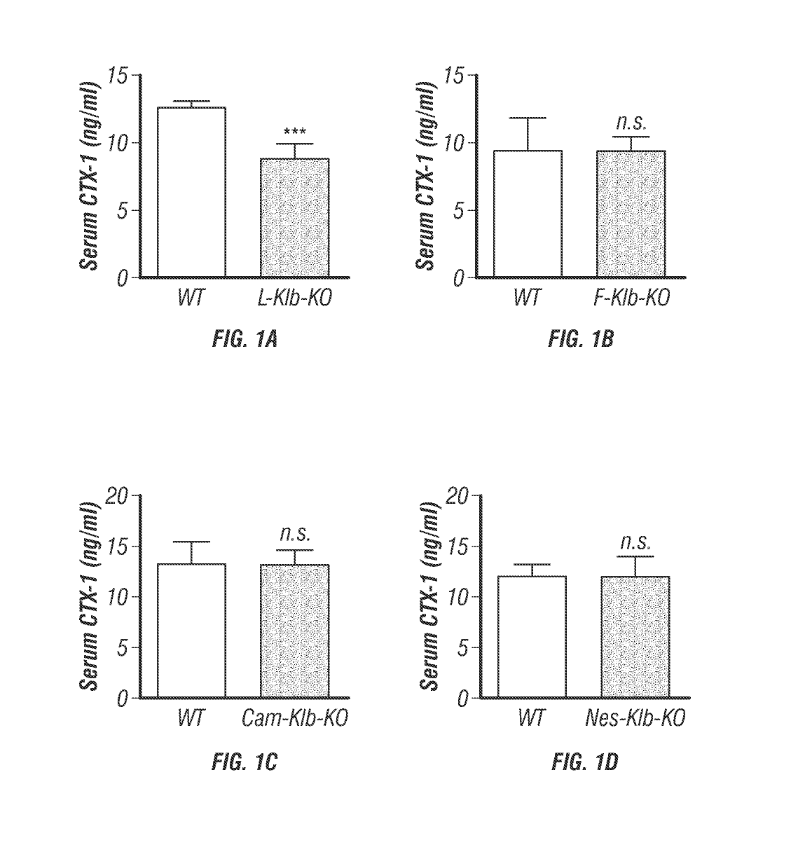

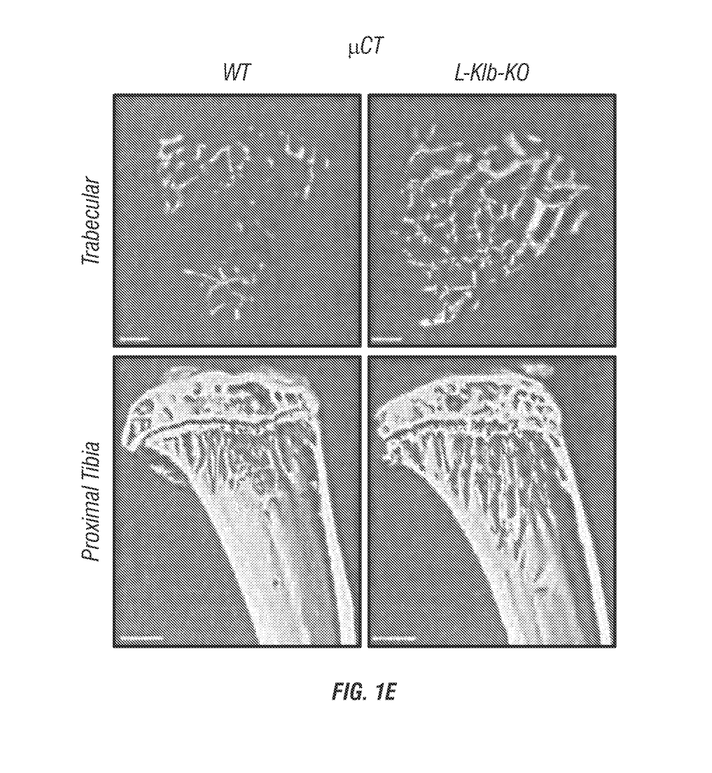

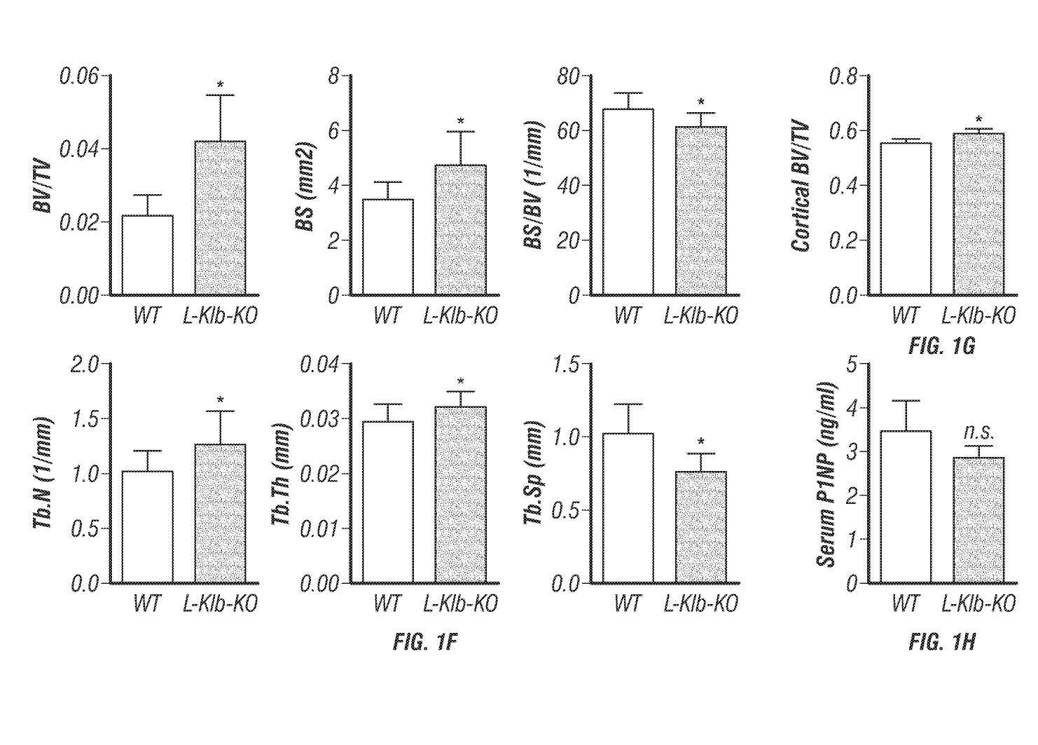

The single-transmembrane protein β-Klotho functions as an essential co-receptor for FGF21 in vitro and in vivo (Ding et al., 2012, Kharitonenkov et al., 2008, Ogawa et al., 2007 and Suzuki et al., 2008). While FGF receptors are widely expressed, the expression of β-Klotho is restricted to just a few FGF21 target tissues, including liver, fat and brain (Fon Tacer et al., 2010). Therefore, tissue-specific β-Klotho deletion using recently established 13-Klotho flox mice represent a powerful approach to dissect the functional requirement of individual tissue as a direct FGF21 target for its physiological and pharmacological actions (Ding et al., 2012).
To generate liver-specific β-Klotho knockout mice (L-Klb-KO), the inventor bred β-Klotho flox mice with albumin-cre mice (Yakar et al., 1999). To generate fat-specific β-Klotho knockout mice (F-Klb-KO), the inventor bred β-Klotho flox mice with ap2-cre mice (Ding et al., 2012). To generate CNS-specific β-Klotho knockout mice, the inventor bred β-Klotho flox mice with CamK-cre (Cam-Klb-KO) (Bookout et al., 2013, Casanove et al., 2001 and Owen et al., 2013) or Nestin-cre (Nes-Klb-KO) (Tronche et al., 1999). ELISA analysis of a bone resorption marker C-terminal telopeptide fragments of the type I collagen (CTX-1) revealed that bone resorption was specifically decreased in the L-Klb-KO mice (FIG. 1A), but unaltered in F-Klb-KO (FIG. 1B), Cam-Klb-KO (FIG. 1C) or Nes-Klb-KO (FIG. 1D).
Micro-computed tomography (μCT) analysis of the proximal tibia revealed that the reduced bone resorption in L-Klb-KO mice resulted in a higher bone mass (FIG. 1E). Quantification of trabecular bone structure and architecture showed that the bone volume/tissue volume ratio (BV/TV) was increased by 95% in L-Klb-KO compared to WT littermate controls, accompanied by 35% greater bone surface (BS) and 9% lower bone surface/bone volume ratio (BS/BV), 24% greater trabecular number (Tb.N), 9% greater trabecular thickness (Tb.Th) and 25% less trabecular separation (Tb.Sp) (FIG. 1F). In agreement, the BV/TV in the cortical bone was also 7% higher in L-Klb-KO mice (FIG. 1G). The bone formation marker N-terminal propeptide of type I procollagen (P1NP) was not significantly altered (FIG. 1H), indicating that liver β-Klotho specifically regulates bone resorption without affecting bone formation under physiological conditions.

### Liver β-Klotho Deletion Abolishes FGF21-Induced Bone Loss but not FGF21-Induced Insulin Sensitization.

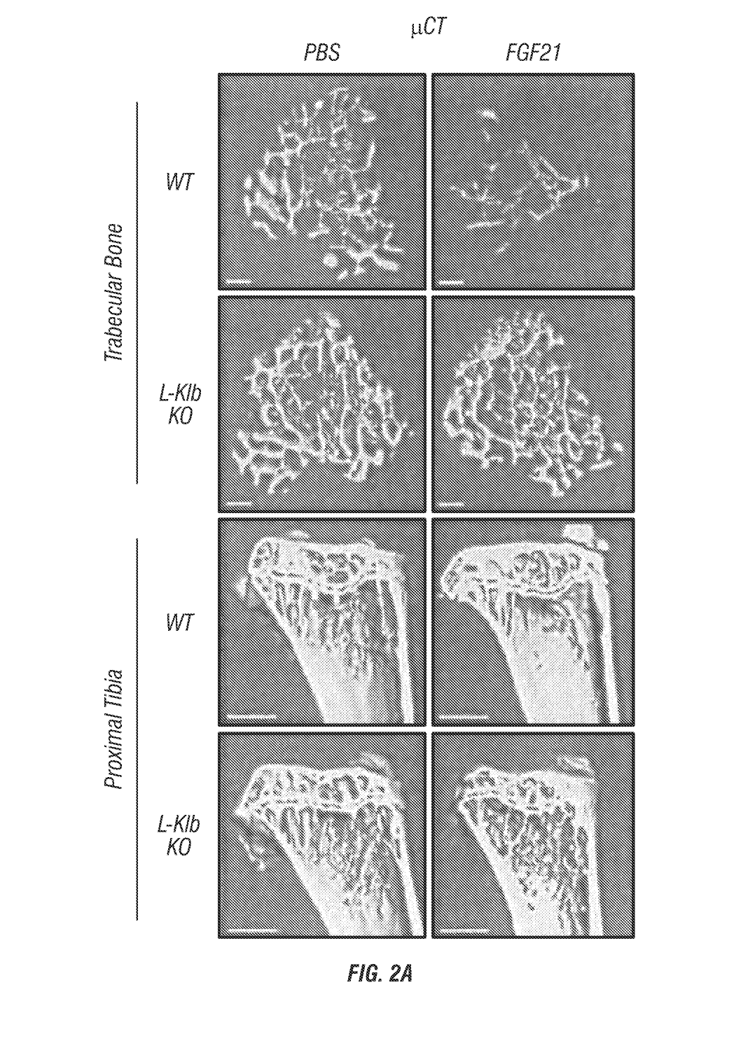

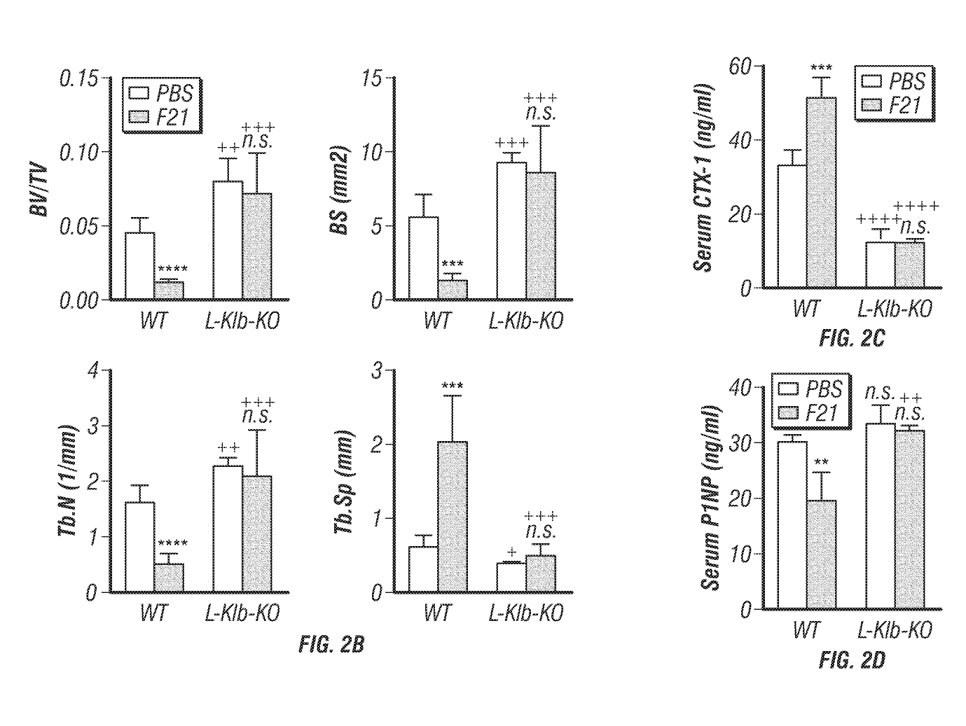

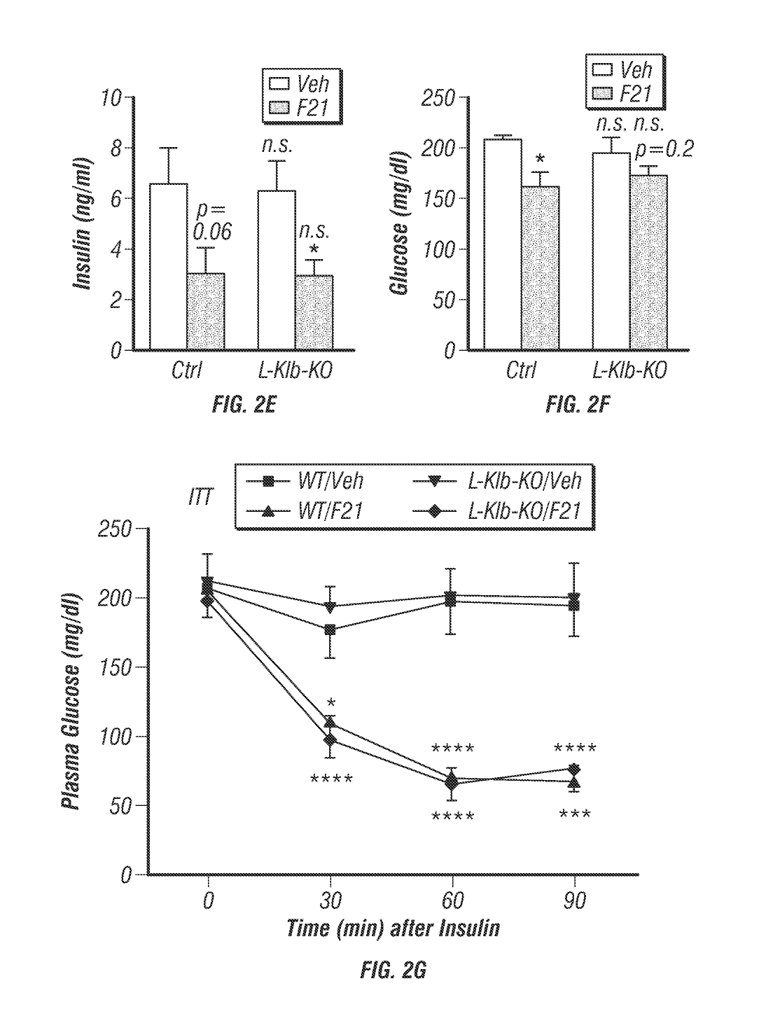

The inventor next investigated whether liver β-Klotho is an essential mediator of FGF21-induced bone loss. L-Klb-KO mice or WT controls (male, 6-month-old at end point) were fed a high-fed-diet for 4.5 months and then treated with recombinant FGF21 (1 mg/kg/day) or vehicle control for the last 14 days. μCT showed that pharmacological FGF21 treatment caused a significant bone loss in WT mice (FIG. 2A) demonstrated by the lower BV/TV (−78%), BS (−77%) and Tb.N (−67%), as well as the higher Tb.Sp (+230%) (FIG. 2B). In contrast, the L-Klb-KO mice were completely resistant to the bone loss (FIGS. 2A-B). In line with these observations, L-Klb-KO mice were also resistant to the FGF21-mediated pharmacological induction of bone resorption (FIG. 2C). Although the physiological basal bone formation was unaltered in L-Klb-KO mice (FIG. 2D), the pharmacological inhibition of bone formation by FGF21 was also prevented in L-Klb-KO mice (FIG. 2D). Interestingly, FGF21-mediated pharmacological reduction of plasma insulin (FIG. 2E) and plasma glucose (FIG. 2F) was intact in L-Klb-KO mice. Moreover, insulin tolerance test showed that FGF21 significantly increased insulin sensitivity in both WT and L-Klb-KO mice to a similar extent (FIG. 2G). Collectively, these results indicate that β-Klotho deletion in the liver specifically abolishes the detrimental pharmacological effects of FGF21 on bone without compromising its beneficial pharmacological effects on glucose metabolism.

### IGFBP1 is an FGF21-Induced Pro-Osteoclastogenic Hepatokine.

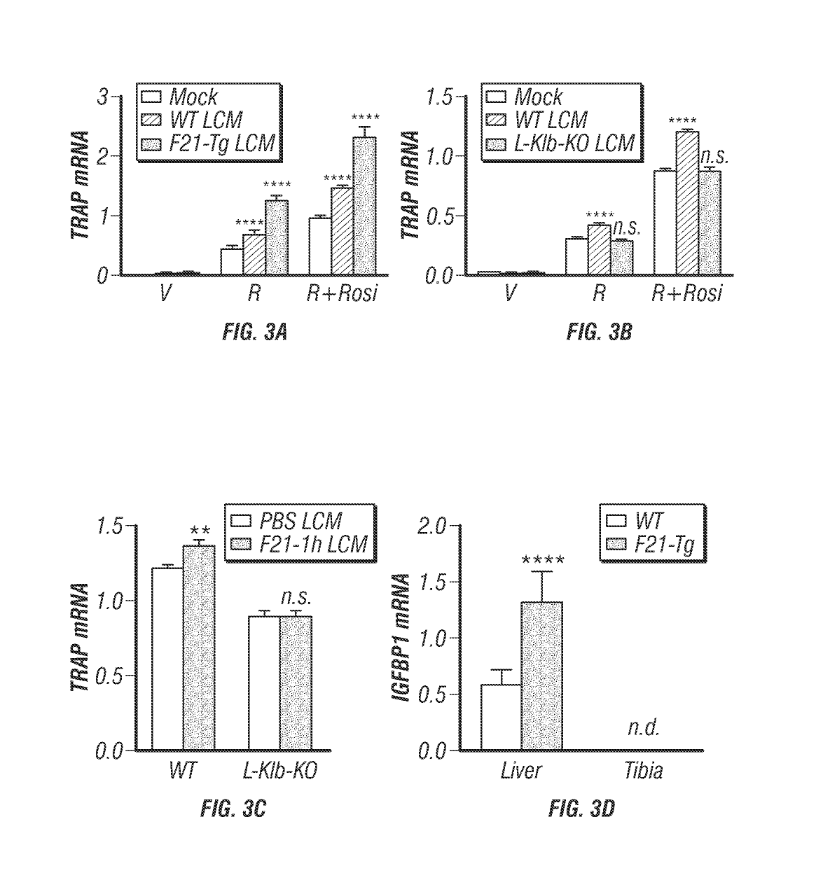

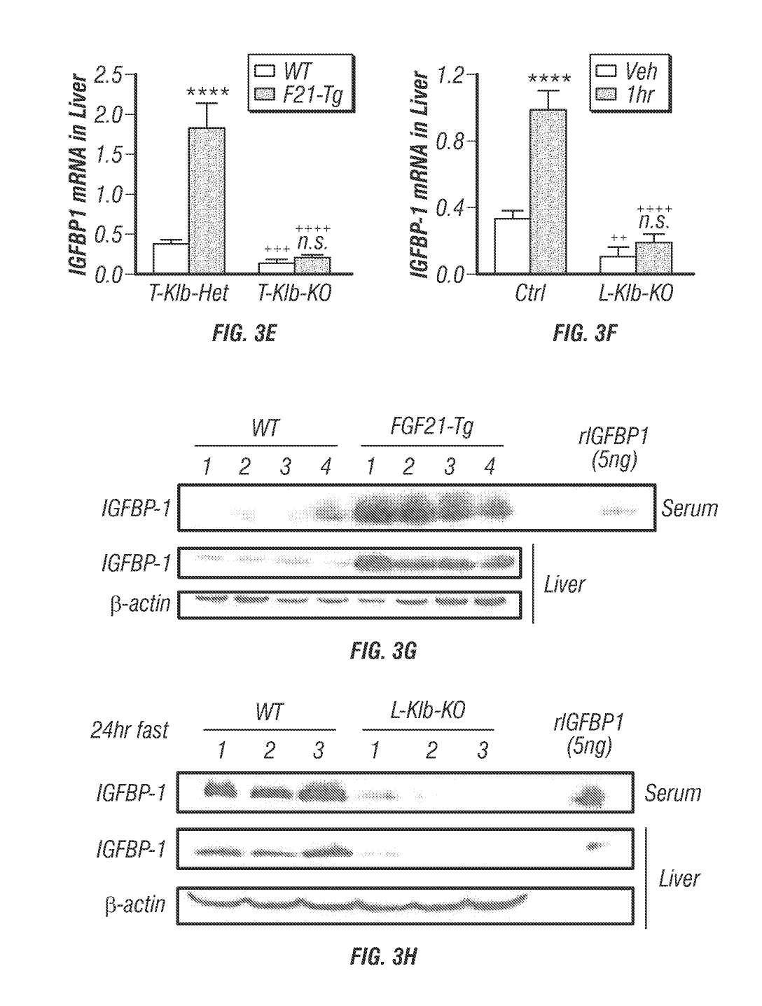

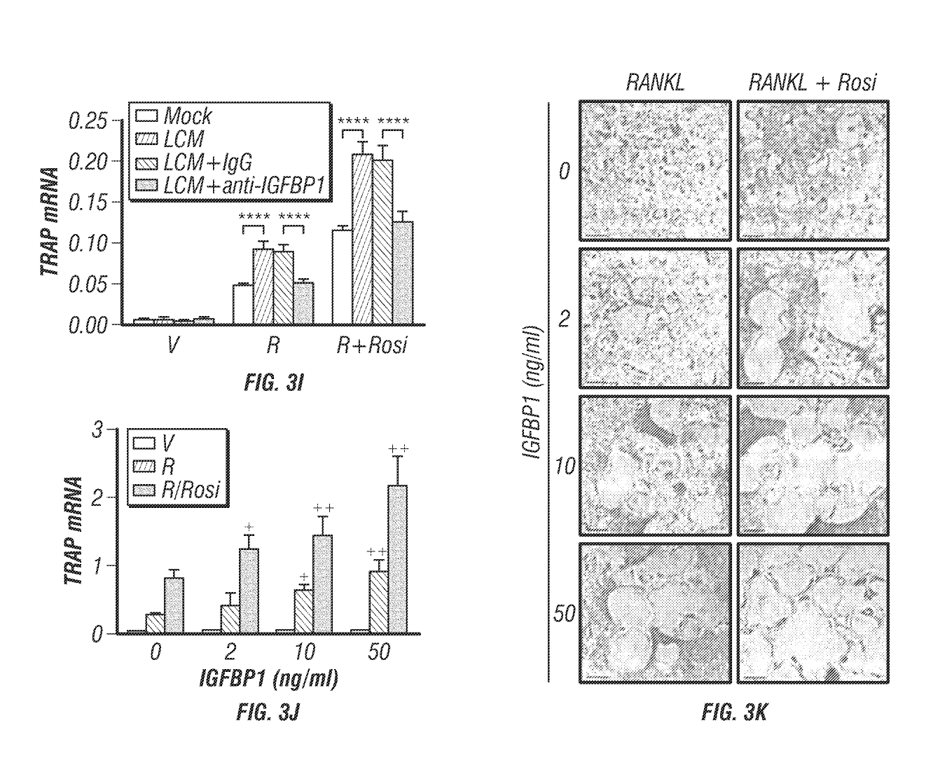

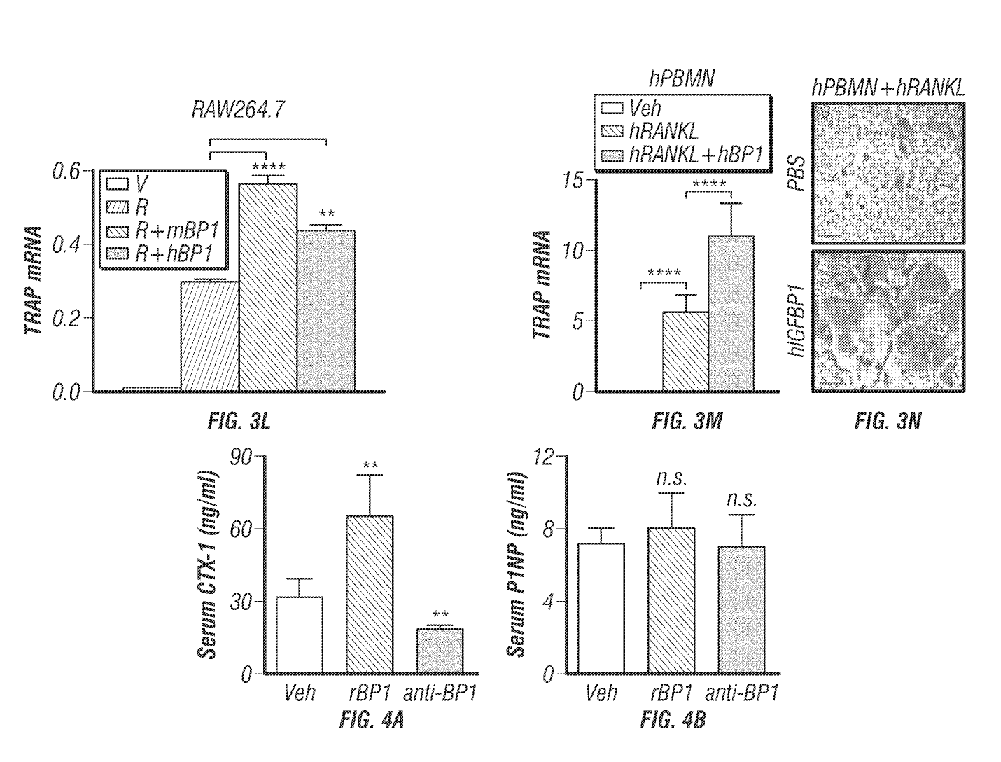

The inventor hypothesized that liver secrets endocrine factor(s) that can directly promote osteoclastogenesis in an FGF21-enhanced and liver-β-Klotho-dependent manner. To test this hypothesis, the inventor collected liver conditioned medium (LCM) from WT, FGF21-Tg or L-Klb-KO mice and determined their effects on RANKL-mediated and rosiglitazone-stimulated osteoclast differentiation from WT bone marrow cells. Compared to mock treatment, osteoclast differentiation was significantly augmented by LCM from WT mice and further enhanced by LCM from FGF21-Tg mice, quantified by the expression of osteoclast marker genes such as TRAP (tartrate-resistant acid phosphatase) (FIG. 3A). The pro-osteoclastogenic activity in WT LCM was absent in the LCM from L-Klb-KO mice (FIG. 3B). Moreover, the LCM from WT mice treated with FGF21 for 1 hr, compared to PBS-treated control mice, also significantly stimulated osteoclast differentiation in a liver-β-Klotho-dependent manner (FIG. 3C). These results indicate that WT liver secrets pro-osteoclastogenic factor(s) in response to physiological levels of FGF21, which is enhanced by FGF21 over-expression or pharmacological FGF21 treatment but abolished by β-Klotho deletion in the liver.
To identify this pro-osteoclastogenic hepatokine, the inventor searched for liver-specific secreted factors that are up-regulated by FGF21. Our previous studies show that chronic and acute FGF21 gain-of-function increases the expression of several genes in the liver including two secreted factors, IGF-1 and IGFBP1 (Inagaki et al., 2008). IGF-1 expression is not liver-specific because it is also abundant in many other tissues including fat and bone; moreover, IGF-1 mainly functions as an autocrine/paracrine factor to increase osteoblast-mediated bone formation (Agnusdei et al., 2005, Belfiore et al., 2009, Giustina et al., 2008, Yakar et al., 2003, Yakar et al., 2005 and Xian et al., 2012). Therefore, the liver-dependence of the bone resorption phenotype and the absence of a bone formation defect in the L-Klb-KO mice argue against IGF-1 as the responsible hepatokine. In contrast, IGFBP1 expression is high and FGF21-inducible in liver but absent in bone (FIG. 3D), thus representing a good candidate for further investigation.

The inventor first examined whether the FGF21 induction of liver IGFBP1 mRNA expression was β-klotho-dependent. Both a chronic FGF21 over-expression in the FGF21-Tg mice (FIGS. 3D-E) and an acute 1 hr FGF21 treatment (FIG. 3F) significantly elevated liver IGFBP1 expression. Both global β-klotho deletion (FIG. 3E) and liver β-klotho deletion (FIG. 3F) reduced basal IGFBP1 expression and attenuated the FGF21 induction of IGFBP1 in the liver. The inventor also examined whether IGFBP1 protein expression and secretion is induced by FGF21. Western blot analyses showed that both liver and serum IGFBP1 protein levels were increased in the FGF21-Tg mice compared to WT control mice (FIG. 3G). Furthermore, both liver and serum IGFBP1 protein levels were reduced in the L-Klb-KO mice compared to WT control mice upon fasting (FIG. 3H), a physiological stimulus of liver FGF21 and IGFBP1 expression (Inagaki et al., 2008).

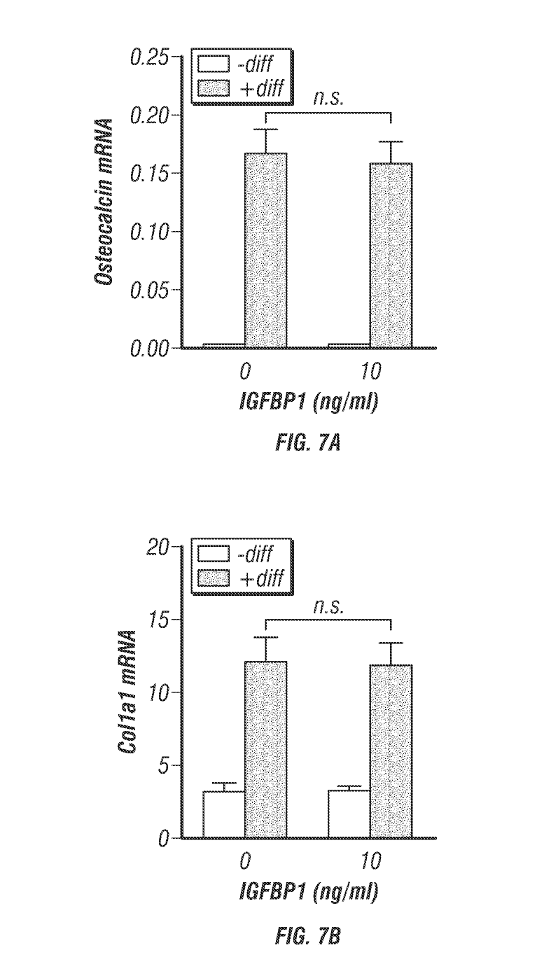

The inventor next investigated whether IGFBP1 is required for the pro-ostoclastogenic activity in WT LCM by testing if the activity can be eliminated by an IGFBP1-blocking antibody. Compared to an IgG control, anti-IGFBP1 completely abolished the osteoclast-enhancing effects of WT LCM (FIG. 3I). This indicates that IGFBP1 is the major pro-osteoclastogenic factor in the liver secretome.
To determine whether IGFBP1 is sufficient to stimulate osteoclastogenesis in the absence of other factors in the liver secretome such as IGF-1, the inventor next treated WT bone marrow osteoclast differentiation cultures with recombinant IGFBP1. IGFBP1 significantly stimulated osteoclast differentiation at a concentration as low as 2 ng/ml, and the effects were enhanced in a dose-dependent manner, demonstrated by an increase in both TRAP induction (FIG. 3J) and the number and size of mature osteoclasts (FIG. 3K). Moreover, RANKL-induced osteoclast differentiation from the RAW264.7 mouse macrophage cell line was also significantly enhanced by both mouse IGFBP1 and human IGFBP1 (FIG. 3L). In contrast, IGFBP1 did not affect bone marrow osteoblast differentiation as the induction of osteoblast markers such as osteocalcin and Collal was unaltered (FIG. 7). This is in agreement with the specific physiological regulation of bone resorption without affecting bone formation in the L-Klb-KO mice (FIG. 1).
To determine whether IGFBP1 also regulates osteoclast differentiation in human, the inventor treated human peripheral blood mononuclear (hPBMN) precursor cells with human RANKL, in the presence of human IGFBP1 or PBS control. Osteoclast differentiation from hPBMN was also significantly augmented by hIGFBP1, shown by the higher TRAP expression (FIG. 3M) and the increased number and size (FIG. 3N) of mature osteoclasts. These results indicate that IGFBP1 promotes osteoclastogenesis in both mice and humans, thus IGFBP1 regulation of bone resorption in mouse models will likely translate to human skeletal physiology and diseases. Collectively, these results show that IGFBP1 is a pro-osteoclastgenic hepatokine that is induced by FGF21 in the liver in a β-klotho-dependent manner and functions as an endocrine hormone upon the skeleton.

### Pharmacological IGFBP1 Manipulations Alter Bone Resorption In Vivo.

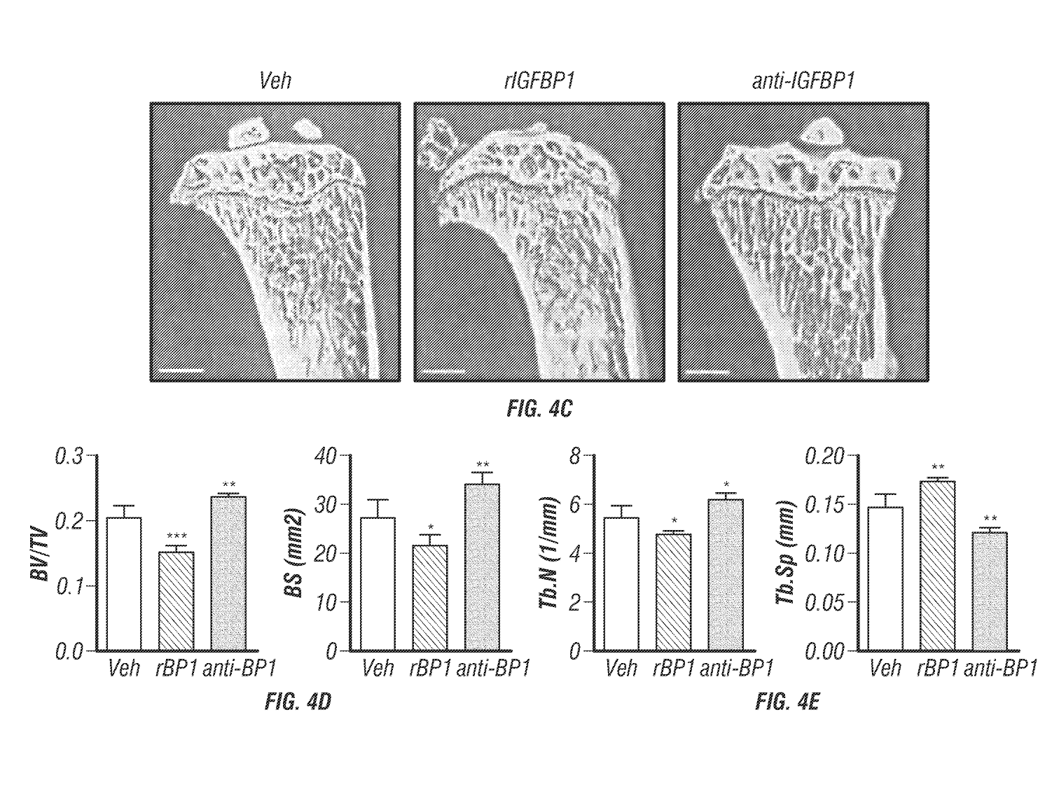

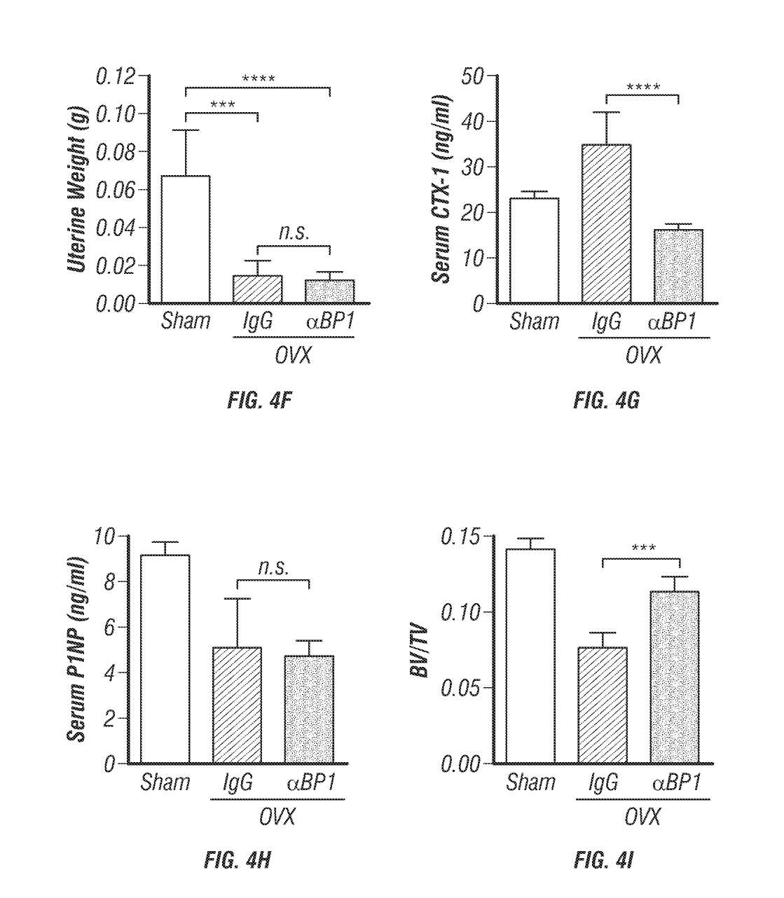

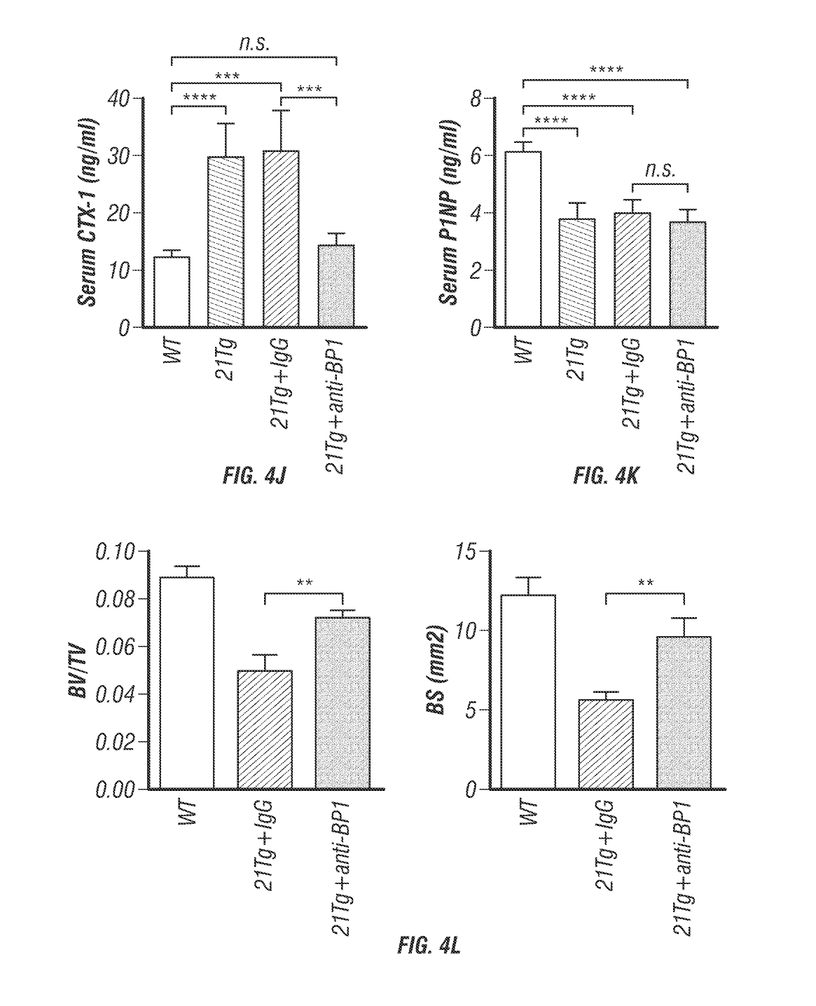

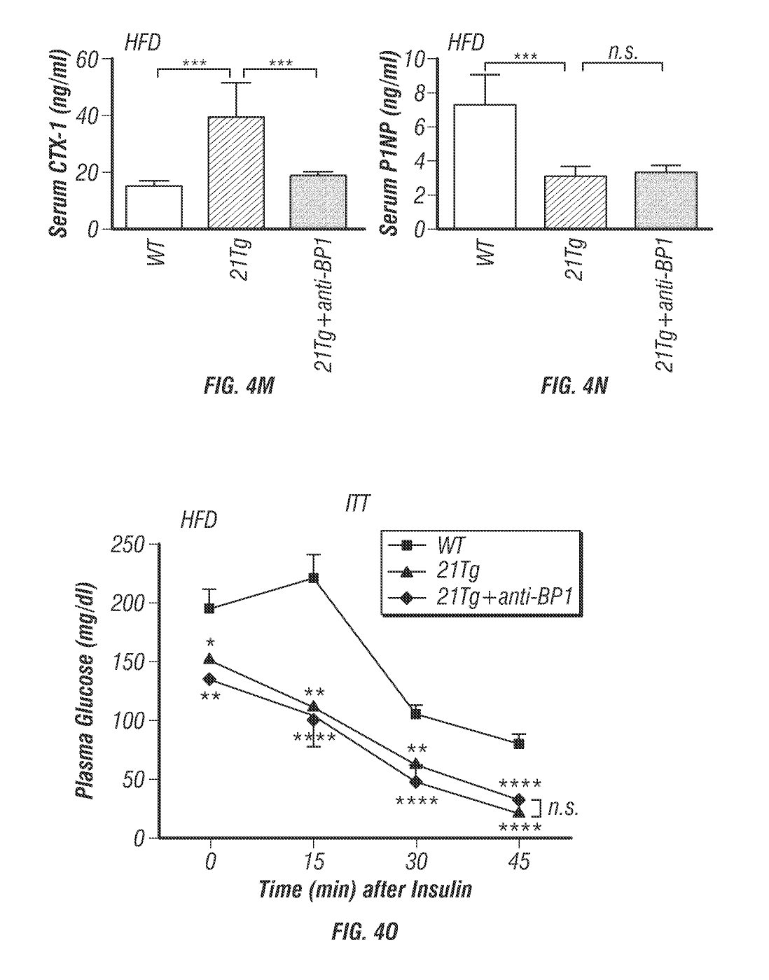

The inventor next investigated whether pharmacological IGFBP1 gain- or loss-of-function regulates bone resorption in vivo. For gain-of-function, the inventor administered recombinant IGFBP1 into WT mice with a daily IP injection at 0.015 mg/kg/day for 14 days. For loss-of-function, she administered an IGFBP1 blocking antibody into WT mice with a daily IP injection at 0.03 mg/kg/day for 14 days. ELISA analyses showed that, compared to vehicle control, serum CTX-1 bone resorption marker was increased by 110% by rIGFBP1 and decreased by 42% by anti-IGFBP1 (FIG. 4A); whereas serum P1NP bone formation marker was unaltered (FIG. 4B). Consequently, μCT imaging revealed that IGFBP1 treatment caused a significant bone loss (FIG. 4C), demonstrated by 26% less BV/TV, 21% less BS, 12% less Tb.N and 18% greater Tb.Sp (FIG. 4D). In contrast, anti-IGFBP1 treatment protected bone leading to a significantly higher bone mass, shown by 16% greater BV/TV, 24% greater BS, 14% greater Tb.N and 18% less Tb.Sp (FIG. 4D).

The inventor next examined whether anti-IGFBP1 treatment can attenuate the bone resorption induced during postmenopausal osteoporosis in a mouse model. To simulate the estrogen loss in postmenopausal women, the inventor performed ovariectomy (OVX) in female WT mice. Three days after surgery, the inventor IP-injected anti-IGFBP1 or an IgG negative control at 0.03 mg/kg and three times per week for 5 weeks. Uterine weight was reduced by ˜80% in all OVX mice compared to sham control mice, indicating effective estrogen depletion (FIG. 4F). Serum CTX-1 was significantly increased by 55% in OVX mice treated with IgG control compared to sham control mice (FIG. 4G). In contrast, the OVX-induced bone resorption was completely abolished by anti-IGFBP1 treatment, which instead led to a 28% reduction in CTX-1 compared to sham controls (FIG. 4G). Although bone formation was unaffected by anti-IGFBP1 treatment (FIG. 4H), OVX-induced bone loss was largely rescued (FIG. 4I). These results suggest that pharmacological IGFBP1 blockade may represent a new therapeutic strategy for the treatment of osteoporosis.

The inventor also tested whether anti-IGFBP1 treatment can ameliorate the bone resorption induced by FGF21 in FGF21-Tg mice. Under chow-diet feeding, anti-IGFBP1 administration (0.03 mg/kg/day for 14 days) effectively reduced serum CTX-1 bone resorption marker in the FGF21-Tg mice to a similar level as in WT control mice (FIG. 4J), without affecting the P1NP bone formation marker (FIG. 4D), leading to a significant rescue of the FGF21-induced bone loss (FIG. 4L). Similarly, under high-fat-diet feeding, anti-IGFBP1 also significantly prevented FGF21 induction of bone resorption (FIG. 4M), without affecting FGF21 reduction of bone formation (FIG. 4N). Importantly, ITT assay showed that the improved insulin sensitivity in FGF21-Tg mice was intact following anti-IGFBP1 treatment (FIG. 4O). These findings indicate that pharmacological IGFBP1 blockade may represent an exciting strategy to prevent the bone loss side effects while retaining the metabolic benefits of FGF21.

### IGFBP1 Potentiates RANKL Signaling by Enhancing Erk-Phosphorylation and NFATc1 Activation.

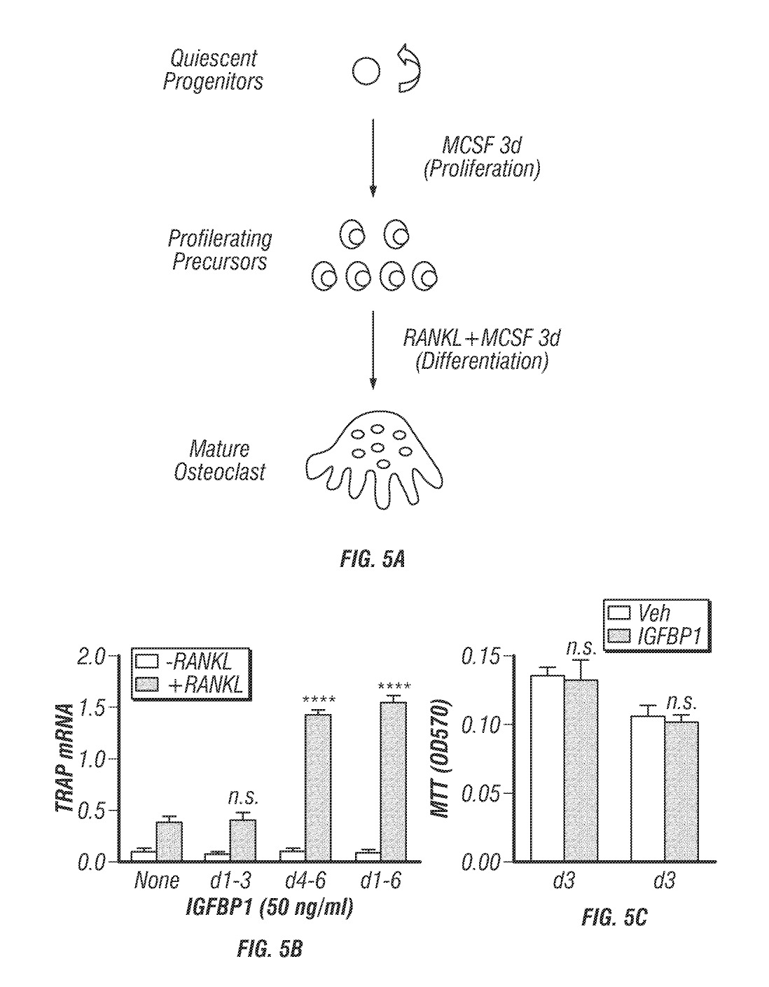

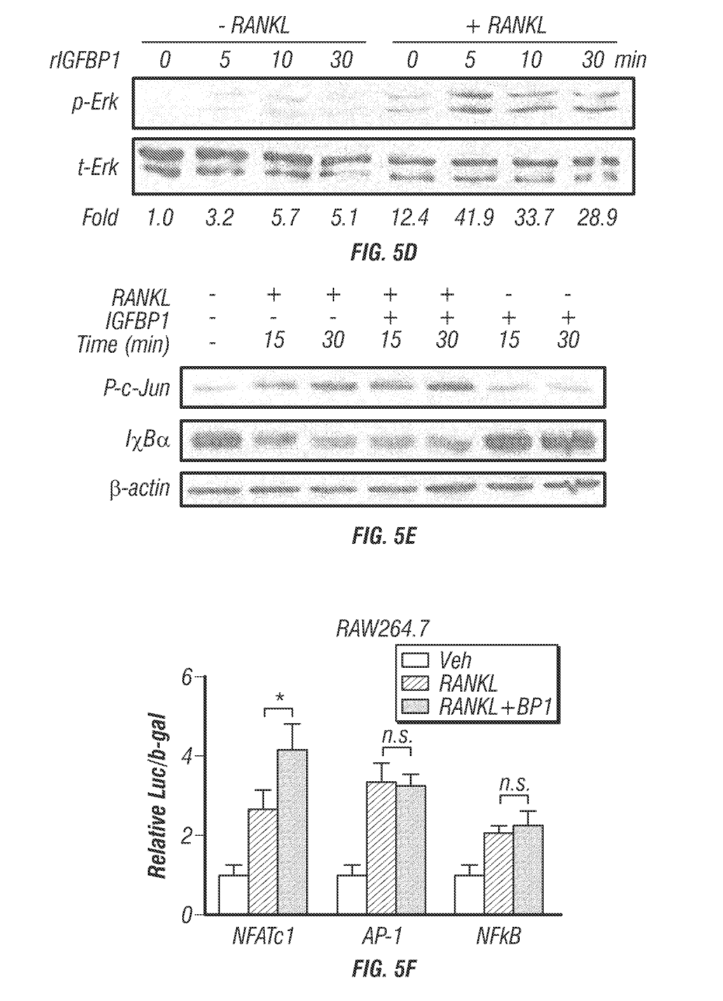

The inventor next investigated the molecular mechanisms for how IGFBP1 enhances osteoclastogenesis. Our bone marrow osteoclast differentiation scheme allowed us to specifically dissect the effects of IGFBP1 on precursor proliferation during the first 3 days of MCSF treatment (d1-3), and osteoclast differentiation during the latter 3 days of RANKL+MCSF treatment (d4-6) (FIG. 5A). When IGFBP1 treatment was limited to d4-6 only, osteoclast differentiation was enhanced to a similar extent as when IGFBP1 treatment was throughout the entire 6 days (d1-6). In contrast, osteoclast differentiation was unaffected when IGFBP1 treatment was limited to d1-3 (FIG. 5B). Consistent with these observations, MTT assay showed that IGFBP1 did not alter cell proliferation on either d3 or d6 (FIG. 5C). Together, these results indicate that IGFBP1 promotes osteoclastogenesis by enhancing RANKL-mediated differentiation without affecting MCSF-mediated precursor proliferation.

The inventor next examined how IGFBP1 potentiates RANKL signaling. She found that IGFBP1 enhanced both basal and RANKL-induced Erk phosphorylation in bone marrow osteoclast differentiation cultures (FIG. 5D). In contrast, IGFBP1 did not alter basal or RANKL-induced c-jun phosphorylation or IκBα degradation (FIG. 5E). To identify which RANKL downstream transcription factor(s) is regulated by IGFBP1 signaling, the inventor transfected RAW267.4 cells with a luciferase reporter driven by the response elements for NFATc1, AP-1 or NFκB. As expected, RANKL treatment activated these endogenous transcription factors, leading to the induction of their corresponding luciferase reporter (FIG. 5F). IGFBP1 co-treatment selectively potentiated the RANKL-induction of NFATc1 reporter, but not AP-1 or NFκB reporters (FIG. 5F). These results indicate that IGFBP1 promotes RANKL-mediated osteoclastogenesis by specifically enhancing Erk phosphorylation and NFATc1 activation.

### IGFBP1 Functions Via RGD Binding to Integrin α5β1 Receptor in the Osteoclast Lineage.

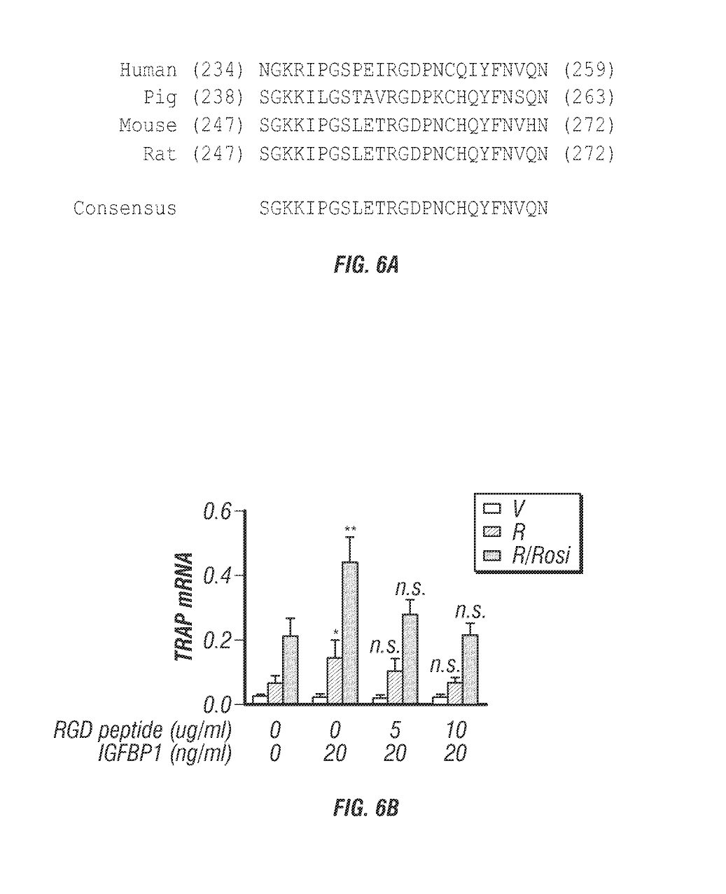

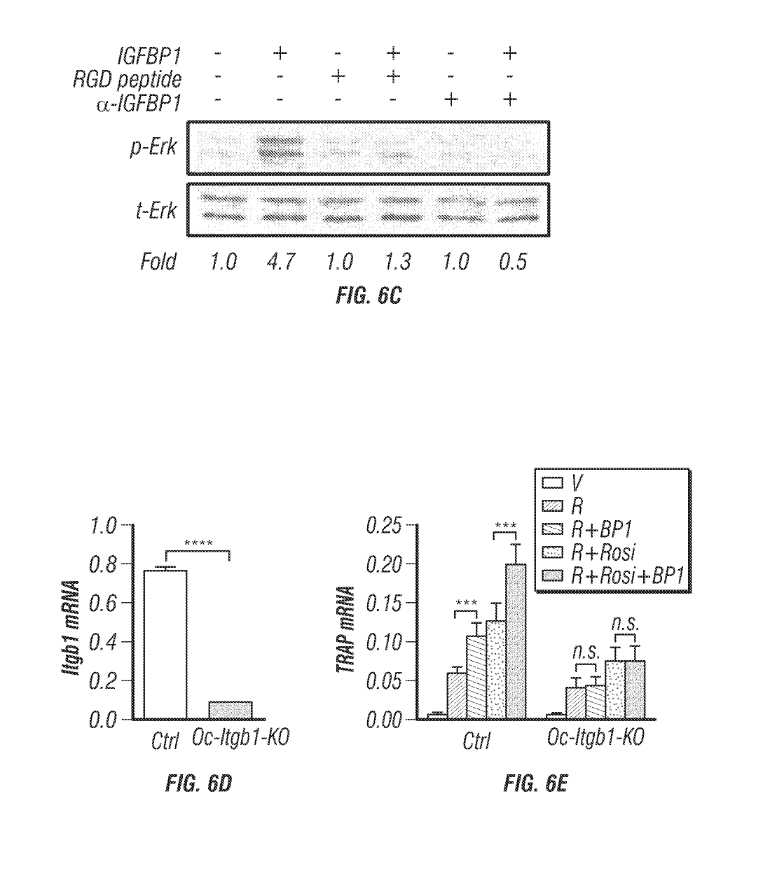

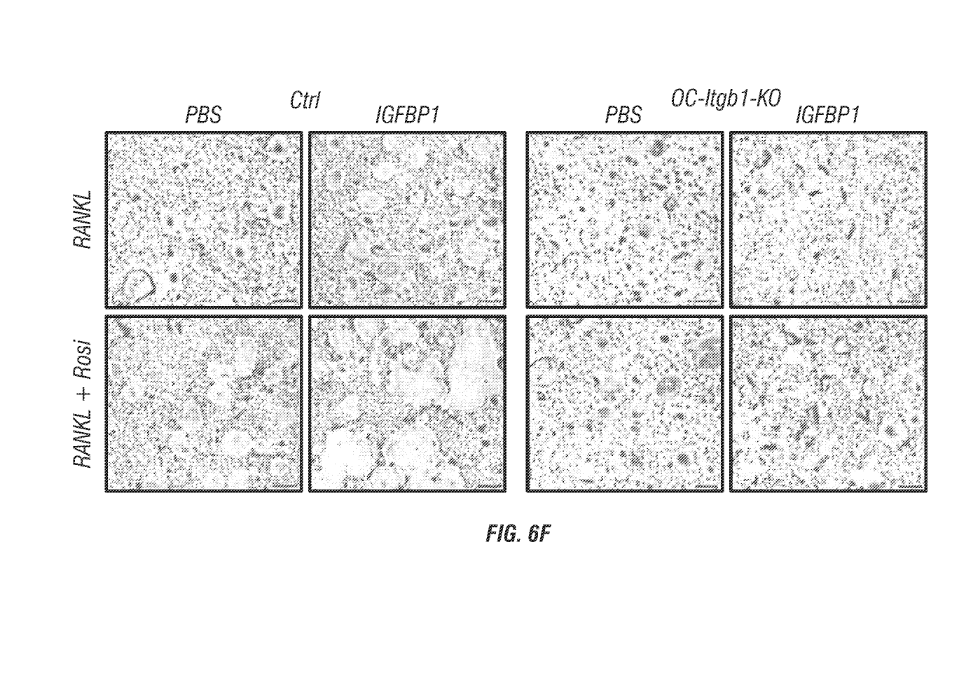

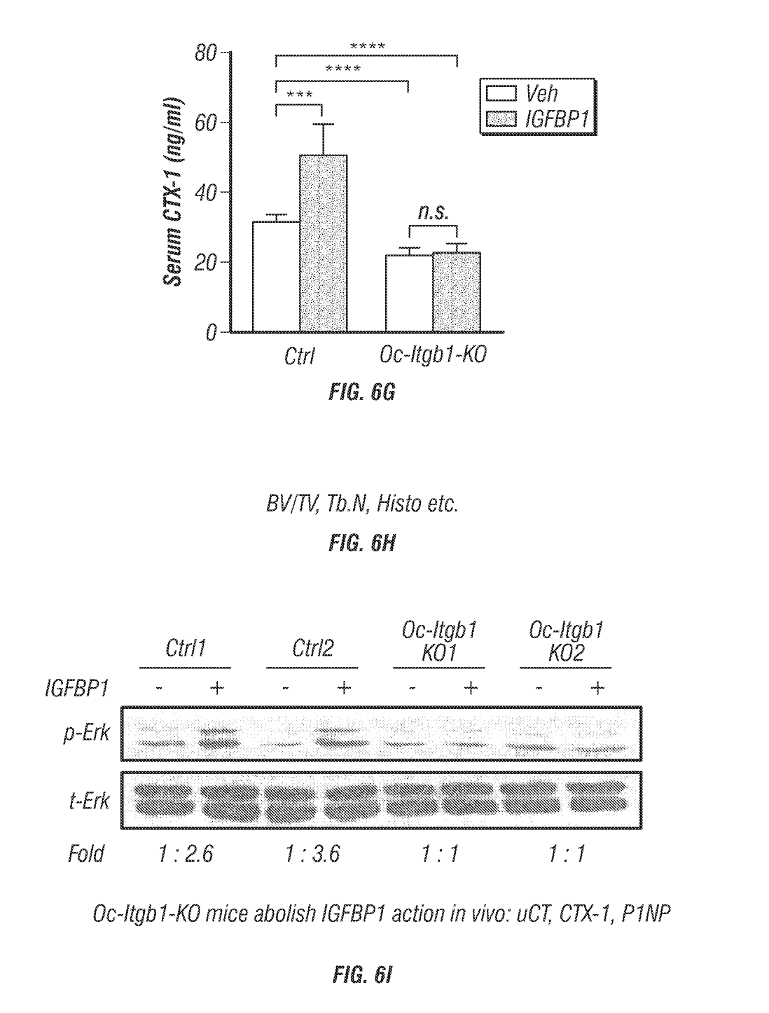

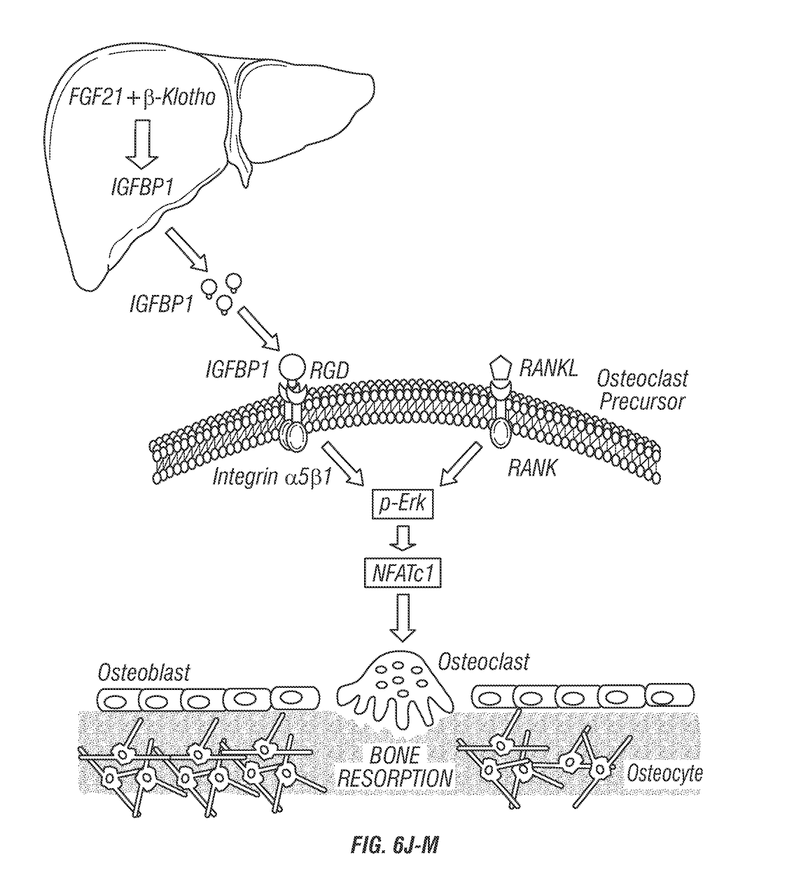

The inventor next set out to identify the IGFBP1 receptor on osteoclast precursors. An RGD (Arginine-Glycine-Aspartic acid) integrin recognition motif in the C-terminus of IGFBP1 is highly conserved among mammals including human, pig, mouse and rat (FIG. 6A), indicating IGFBP1 may function as a ligand for integrin receptors. A previous study using Chinese hamster ovary (CHO) cells reports that integrin α5β1 is the only cell surface receptor that can bind to IGFBP1 in an RGD-dependent but IGF1-independent fashion (Jones et al., 1993). The inventor found that both IGFBP1 stimulation of osteoclastogenesis and IGFBP1 induction of Erk phosphorylation were abolished by the addition of a synthetic RGD-containing competing peptide to WT bone marrow osteoclast differentiation culture (FIGS. 6B-C). These findings indicate that the RGD binding motif in IGFBP1 is functionally required; suggesting that integrin α5β1 may be the IGFBP1 receptor for its pro-osteoclastogenic activity. Integrin α5 can partner with several integrin β subunits including integrin β3 that has been shown to regulate osteoclastogenesis (Novack et al., 2008). Therefore, the specificity of IGFBP1 binding likely resides in integrin β1 (Itgb1).
To determine the in vivo requirement of Itgb1 for IGFBP1 stimulation of osteoclastogenesis and bone resorption, the inventor generated osteoclast-specific Itgb1 knockout mice (Oc-Itgb1-KO) by breeding Itgb1 flox mice (Raghavan et al., 2000) with lysozyme-cre mice (Clausen et al., 1999). Ex vivo bone marrow osteoclast differentiation assay showed that Itgb1 expression was reduced by 88% in the Oc-Itgb1-KO cultures, indicating efficient Itgb1 deletion (FIG. 6D). Compared to WT control cultures, Oc-Itgb1-KO cultures were completely resistant to IGFBP1 potentiation of osteoclast differentiation, but still sensitive to RANKL and rosiglitazone induction of osteoclast differentiation (FIGS. 6E-F).
The inventor then analyzed in vivo consequences by comparing Oc-Itgb1-KO mice with littermate WT control mice, treated with IGFBP1 or vehicle control (0.015 mg/kg/day for 14 days). Consistent with the ex vivo observations, the IGFBP1-induced bone resorption was completely abolished in Oc-Itgb1-KO mice (FIG. 6G), indicating that Itgb1 is required for the pharmacological effects of IGFBP1. Moreover, the basal bone resorption was also significantly lower in Oc-Itgb1-KO mice compared to control mice (FIG. 6G), indicating that Itgb1 is also required for the physiological regulation by IGFBP1. Consequently, Oc-Itgb1-KO mice had higher basal bone mass and were refractory to IGFBP1-induced bone loss (FIG. 6H). Mechanistically, IGFBP1 induction of Erk-phosphorylation was also abolished in Oc-Itgb1-KO osteoclast precursors (FIG. 6I). These results indicate that Itgb1 is the receptor for IGFBP1 that mediates its pro-osteoclastogenic function.

### Osteoclastic Integrin β1 Deletion Abolishes FGF21-Induced Bone Loss but not FGF21-Induced Insulin Sensitization.

To further examine whether the IGFBP1-Itgb1 signaling pathway is required for FGF21-induced bone resorption and bone loss, the inventor bred Oc-Itgb1-KO mice with FGF21-Tg mice. As expected, bone resorption was elevated in FGF21-Tg mice compared to WT littermate controls (FIG. 6J), leading to a lower bone mass (FIG. 6K). In contrast, bone resorption in the FGF21-Tg/Oc-Itgb1-KO compound mutants remained low at a similar level as in the WT controls (FIG. 6J), resulting in a normal bone mass as in WT controls (FIG. 6K). Importantly, ITT assays reveal that both FGF21-Tg and the FGF21-Tg/Oc-Itgb1-KO compound mutants exhibited higher insulin sensitivity compared to WT controls (FIG. 6L). These data indicate that Itgb1 deletion in the osteoclast lineage specifically abolishes FGF21-induced bone loss side effects while retaining FGF21-mediated insulin sensitization metabolic benefits. Together, these findings further support that Itgb1 functions as an IGFBP1 receptor in the osteoclast lineage to mediate the resorption-enhancing effects of the liver-bone hormonal relay orchestrated by the FGF21-IGFBP1 axis.
In summary, this study has uncovered a previously unrecognized liver-bone endocrine relay, as well as a key mechanism for FGF21-induced bone resorption. Physiological or pharmacological elevation of FGF21 induces the expression and secretion of IGFBP1 from the liver into circulation in a β-klotho dependent manner. In turn, IGFBP1 acts directly on the osteoclast precursors in bone by binding to integrin β1 receptor via its RGD motif. Consequently, IGFBP1 stimulates osteoclast differentiation and bone resorption by potentiating RANKL-induced Erk phosphorylation and NFATc1 activation, leading to lower bone mass (FIG. 6M). Therefore, the inventor has identified IGFBP1 as a novel endocrine hormone that is secreted from the liver in response to metabolic cues to regulate bone resorption and skeletal homeostasis. Importantly, the inventor has found that an anti-IGFNP1 antibody can suppress bone resorption and increase bone mass, revealing pharmacological IGFBP1 blockade may represent a novel strategy for the treatment of osteoporosis.
Furthermore, the inventor have identified liver β-klotho and the IGFBP1-Itgb1 axis as essential and specific mediators of FGF21-induced bone resorption, because liver β-klotho deletion, IGFBP1 blockade or osteoclastic Itgb1 deletion all confer resistance to FGF21-induced bone loss without compromising its insulin-sensitizing benefits. This is consistent with our recent reports that the metabolic benefits of FGF21 are mainly mediated by β-Klotho in the brain and adipose tissue (Ding et al., 2012 and Bookout et al, 2013). Thus, our findings suggest that pharmacological IGFBP1 blockade may also represent a potential avenue to prevent the detrimental skeletal effects of FGF21 while preserving its beneficial metabolic actions.
Several human diseases including osteoporosis and diabetes are associated with higher serum IGFBP1 levels and at the same time bone loss (Jehle et al., 2003, Salminen et al., 2008, Miao et al., 2005, Schwartz et al., 2001, Ruan and Lai 2010, Rosen 2008 and Moyer-Mileur et al., 2008). Interestingly, insulin sensitizers such as thiazolidinedione and FGF21 also simultaneously elevate serum IGFBP1 (Inagaki et al., 2008, Belli et al., 2004 and Seto-Young et al., 2005) and reduce bone mass (Wei et al., 2012, Home et al., 2009, Kahn et al., 2006, Kahn et al., 2008, Zinman et al., 2010 and Bilezikian et al., 2013). Nonetheless, the functional significance and molecular mechanisms underlying IGFBP1 actions in these clinical observations have been long elusive. This study challenges the dogma that IGFBP1 regulates physiology simply as an IGF1 binder by demonstrating that it is itself an endocrine hormone. A quarter of century after its cloning, IGFBP1 is now recognized as a metabolic signal sent from the liver to the bone to directly stimulate osteoclastogenesis and bone resorption via the receptor integrin β1. Provocatively, the discovery of IGFBP1 as a pro-osteoclastogenic hepatokine opens an exciting new path to the fundamental understanding of the physiological and pathological connection between energy metabolism and skeletal homeostasis, as well as better pharmacological treatments of bone and metabolic disorders.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### VII. References

The following references, to the extent that they provide exemplary procedural or other details supplementary to those set forth herein, are specifically incorporated herein by reference.
“Antibodies: A Laboratory Manual,” Cold Spring Harbor Press, Cold Spring Harbor, N.Y., 1988.
Agnusdei et al., J Endocrinol Invest 28:32-36, 2005.
Ausubel et al., Current Protocols in Molecular Biology, Greene Publishing Associates and Wiley Interscience, N. Y., 1994.
Beidler et al., J. Immunol., 141(11):4053-4060, 1988.
Belfiore et al., Endocr Rev 30:586-623, 2009.
Belli et al., Fertility and sterility, 81:624-629, 2004.
Berglund et al., Endocrinology, 150:4084-4093, 2009.
Bilezikian et al., The Journal of clinical endocrinology and metabolism 98:1519-1528, 2013.
Bookout et al., Nature medicine, 2013.
Campbell, In: Monoclonal Antibody Technology, Laboratory Techniques in Biochemistry and Molecular Biology, Vol. 13, Burden and Von Knippenberg, Eds. pp. 75-83, Amsterdam, Elsevier, 1984.
Clausen et al., Transgenic Res 8:265-277, 1999.
Coskun et al., Endocrinology, 149:6018-6027, 2008.
Costa et al., Mol. Cell. Biol., 8:81-90, 1988.
Ding et al., Cell Metab 16:387-393, 2012.
Doolittle and Ben-Zeev, Methods Mol. Biol., 109:215-237, 1999.
EP Application 125,023
EP Application 171,496
EP Application 173,494
EP Application 184,187
Fon Tacer et al., Mol Endocrinol 24:2050-2064, 2010.
Gefter et al., Somatic Cell Genet., 3:231-236, 1977.
Giustina et al., Endocr Rev 29:535-559, 2008.
Goding, In: Monoclonal Antibodies: Principles and Practice, 2d ed., Orlando, Fla., Academic Press, 60-61, 65-66, 71-74, 1986.
Goetz et al., Mol Cell Biol 27:3417-3428, 2007.
Greene et al., Immunology Today, 10:272, 1989.
Harland and Weintraub, J. Cell Biol., 101(3):1094-1099, 1985.
Home et al., Lancet 373:2125-2135, 2009.
Inagaki et al., Cell Metab 8:77-83, 2008.
Jehle et al., Eur J Intern Med 14:32-38, 2003.
Jones et al., Nature, 321:522-525, 1986.
Jones et al., Proceedings of the National Academy of Sciences of the United States of America 90:10553-10557, 1993.
Kahn et al., Diabetes Care 31:845-851, 2008.
Kahn et al., N Engl J Med 355:2427-2443, 2006.
Kharitonenkov et al., J Cell Physiol 215:1-7, 2008.
Kharitonenkov et al., J Clin Invest, 115:1627-1635, 2005.
Kohler and Milstein, Eur. J. Immunol., 6:511-519, 1976.
Kohler and Milstein, Nature, 256:495-497, 1975.
Leibowitz et al., Diabetes, 48(4):745-753, 1999.
Miao et al., Diabetes Care 28:2850-2855, 2005.
Morrison, Science, 229(4719):1202-1207, 1985.
Moyer-Mileur et al., Journal of bone and mineral research: the official journal of the American Society for Bone and Mineral Research, 23:1884-1891, 2008.
Novack et al., Annu Rev Pathol 3:457-484, 2008.
Ogawa et al., Proc Natl Acad Sci USA 104:7432-7437, 2007.
Owen et al., Nature medicine, 2013.
PCT Application PCT/US86/02269
PCT Application WO 86/01533
Posner et al., Hybridoma 6:611-625, 1987.
Potthoff et al., Genes Dev, 2012.
Raghavan et al., The Journal of cell biology 150:1149-1160, 2000.
Rosen, Journal of bone and mineral research: the official journal of the American Society for Bone and Mineral Research 23:1881-1883, 2008.
Ruan and Lai, Acta Diabetol 47:5-14, 2010.
Salminen et al., Osteoporosis international: a journal established as result of cooperation between the European Foundation for Osteoporosis and the National Osteoporosis Foundation of the USA 19:201-209, 2008.
Sambrook et al., In: Molecular cloning: a laboratory manual, 2^{nd} Ed., Cold Spring Harbor Laboratory Press, Cold Spring Harbor, N.Y., 1989.
Schwartz et al., The Journal of clinical endocrinology and metabolism 86:32-38, 2001.
Seto-Young et al., The Journal of clinical endocrinology and metabolism 90:6099-6105, 2005.
Shaw et al., J. Natl. Cancer Inst., 80(19):1553-1559, 1988.
Sun et al., J. Steroid Biochem., 26(1):83-92, 1987.
Suzuki et al., Mol Endocrinol 22:1006-1014, 2008.
Tronche et al., Nature genetics 23:99-103, 1999.
U.S. Pat. No. 4,196,265
U.S. Pat. No. 4,680,338
U.S. Pat. No. 4,816,567
U.S. Pat. No. 4,867,973
U.S. Pat. No. 4,879,236
U.S. Pat. No. 5,141,648
U.S. Pat. No. 5,856,456
U.S. Pat. No. 5,871,986
U.S. Pat. No. 5,880,270
Verhoeyen et al., Science, 239(4847):1534-1536, 1988.
Wan et al., Nat Med 13:1496-1503, 2007.
Wawrzynczak & Thorpe, Cancer Treat Res., 37:239-51, 1988.
Wei et al., Cell Metab 11:503-516, 2010.
Wei et al., Proc Natl Acad Sci USA 109:3143-3148, 2012.
Wood et al., J. Clin. Lab. Immunol., 17(4):167-171, 1985.
Yakar and Rosen, Exp Biol Med (Maywood) 228:245-252, 2003.
Yakar et al., Endocr Dev 9:11-16, 2005.
Yakar et al., Proc Natl Acad Sci USA 96:7324-7329, 1999.
Zinman et al., J Clin Endocrinol Metab 95:134-142, 2010.

What is claimed:

1. A method of treating menopause-induced bone loss in a subject comprising administering to a subject in need thereof an anti-IGFBP1 antibody.

2. The method of claim 1, wherein said antibody is part of a polyclonal antibody preparation.

3. The method of claim 1, wherein said antibody is a monoclonal antibody.

4. The method of claim 3, wherein said antibody is a humanized antibody, a chimeric antibody, an antibody fragment, a bispecific antibody or a single chain antibody.

5. The method of claim 1, wherein said antibody is administered systemically.

6. The method of claim 1, wherein said antibody is administered local or regional to a site of bone loss or potential bone loss.

7. The method of claim 1, wherein said subject is a human subject.

8. The method of claim 1, wherein said subject is a non-human animal subject.

9. The method of claim 1, menopause is age-related.

10. The method of claim 1, menopause is surgery-related.

11. A method of treating a subject for bone resorption induced by FGF21 therapy comprising administering to a subject in need thereof an anti-IGFBP1 antibody.

12. The method of claim 11, wherein said antibody is part of a polyclonal antibody preparation.

13. The method of claim 11, wherein said antibody is a monoclonal antibody.

14. The method of claim 13, wherein said antibody is a humanized antibody, a chimeric antibody, an antibody fragment, a bispecific antibody or a single chain antibody.

15. The method of claim 11, wherein said antibody is administered systemically.

16. The method of claim 11, wherein said antibody is administered local or regional to a site of bone resorption or potential bone resorption.

17. The method of claim 11, wherein said subject is a human subject.

18. The method of claim 11, wherein said subject is a non-human animal subject.

19. The method of claim 11, wherein said antibody is co-administered with FGF21.

20. The method of claim 11, wherein said antibody is administered before or after FGF21.

21. A method of treating FGF21-induced bone loss/resorption comprising administering to a subject in need thereof an integrin β-1 inhibitor.

22. The method of claim 21, wherein said FGF21-induced bone loss/resorption is menopause-related.

23. The method of claim 21, wherein said FGF21-induced bone loss/resorption is FGF21 therapy-related.

24. The method of claim 13, wherein said inhibitor is an anti-integrin β-1 antibody, an integrin β-1 siRNA, an integrin β-1 fragment or sharpin.

25. The method of claim 21, wherein said inhibitor is administered systemically.

26. The method of claim 21, wherein said inhibitor is administered local or regional to a site of bone loss/resorption or potential bone loss/resorption.

27. The method of claim 21, wherein said subject is a human subject.

28. The method of claim 21, wherein said subject is a non-human animal subject.

29. The method of claim 23, wherein said inhibitor is co-administered with FGF21.

30. The method of claim 23, wherein said inhibitor is administered before or after FGF21.


SEQUENCE LISTING (5 sequences)

SEQ ID NO: 1 - protein, 25 aa, Homo sapiens
NGKRIPGSPEIRGDPNCQIYFNVQN

SEQ ID NO: 2 - protein, 25 aa, Sus scrofa
SGKKILGSTAVRGDPKCHQYFNSQN

SEQ ID NO: 3 - protein, 25 aa, Mus musculus
SGKKIPGSLETRGDPNCHQYFNVHN

SEQ ID NO: 4 - protein, 25 aa, Rattus norvegicus
SGKKIPGSLETRGDPNCHQYFNVQN

SEQ ID NO: 5 - protein, 25 aa, Artificial
  Synthetic peptide
SGKKIPGSLETRGDPNCHQYFNVQN